(19) United States
(12) Patent Application Publication
(10) Pub. No.: US 2025/0367438 A1
(43) Pub. Date: Dec. 4, 2025

(54) SURGICAL PROCEDURE TO IMPLANT AN ELECTRODE ASSEMBLY FOR PHRENIC NERVE STIMULATION TO TREAT SLEEP APNEA

(71) Applicant: Lunair Medical, Inc., Minneapolis, MN (US)

(72) Inventors: Edward RUPPLE, New York, NY (US); Mark GELFAND, New York, NY (US); Maria SUURNA, Platation, FL (US); Benedict KINNY-KOSTER, Minneapolis, MN (US)

(21) Appl. No.: 19/108,127

(22) PCT Filed: Aug. 30, 2023
(86) PCT No.: PCT/US2023/073150

§ 371 Date: Feb. 28, 2025

Related U.S. Application Data
(60) Provisional application No. 63/374,075, filed on Aug. 31, 2022.

(51) Int. Cl. A61N 1/05; A61B 17/32; A61B 17/3209; A61N 1/372

(52) U.S. Cl. CPC A61N 1/0553; A61B 17/32093; A61N 1/37247; A61B 2017/320044

(57) ABSTRACT

A delivery tool configured to surgically position an electrode assembly on a patient's anterior scalene muscle and phrenic nerve, the delivery tool including: an electrode carrying portion comprising a receiving space with at least one aperture in a floor of the receiving space, the receiving space being configured to hold an electrode assembly; a lead carrying portion extending from the electrode carrying portion, the lead carrying portion being configured to hold a lead connected to the electrode assembly; and a securing device configured to hold the electrode assembly in place relative to a stimulation target when the delivery tool is inserted into a patient's body, the securing device being further configured to allow the electrode to be repositioned after the electrode assembly has been held in place by the securing device.

### FIELD OF INVENTION

The field of the invention is phrenic nerve stimulation to treat sleep apnea and, particularly, treating sleep apnea by surgically implanting an electrode assembly to stimulate the right, left or both phrenic nerves.

### BACKGROUND

Sleep apnea is a common breathing disorder. A patient suffering sleep apnea repeatedly stops and starts breathing while asleep. These interruptions cause oxygen deprivation in the body through decreased oxygen uptake by the lungs. Sleep apnea often interrupts the patient's sleep cycle, prevents the patient from getting proper rest, and causes the patient to snore loudly. Sleep apnea may increase the risk that a patient will suffer high blood pressure, congestive heart failure and other cardiac related ailments.

Obstructive sleep apnea (OSA) is a common form of sleep apnea in which a patient's airway collapses and prevents air flow to the lungs. Central sleep apnea (CSA) is another form of sleep apnea in which the brain fails to send proper signals to the diaphragm muscle that contracts and expands the lungs to promote breathing.

An approach to treating OSA and/or CSA is to artificially stimulate the phrenic nerve to cause the diaphragm to contract to initiate respiration in a breathing cycle or contract further than would occur during a natural expansion of the lungs. The right-sided phrenic nerve motorically innervates the right part of the diaphragm and the left-sided phrenic nerve motorically innervates the left part of the diaphragm. Sleep apnea treatment may be effective either by unilateral electric stimulation (right or left phrenic nerve) or bilateral electric stimulation (both sides). Artificially stimulating the phrenic nerve may substitute for the brain stimulating the phrenic nerve in the case of CSA. Stimulation of the phrenic nerve may also be effective to treat OSA because contraction of the diaphragm generates traction on the airway and pulls open the airway obstructed due to OSA.

To stimulate the phrenic nerve, an electrode assembly is implanted in the patient near the right or left phrenic nerve. The electrode assembly may be surgically implanted through an incision in the neck by using a cervical surgery approach. In contrast, a thoracic surgical approach is through the chest cavity. Thoracic surgery is an invasive surgery creating an opening through the chest cavity and requiring general anesthesia. Nevertheless, in the past, more invasive thoracic surgery was preferred because of the challenges of the cervical approach that resulted in frequent device failures.

The anatomy of the patient's neck poses unique challenges that are not experienced when positioning electrodes in other locations. For example, the patient's muscles, bones, blood vessels and nerves are tightly located in the neck and move relative to each other. Such movement can cause an unsecured electrode to move relative to the phrenic nerve. When the electrode moves relative to the phrenic nerve, the impedance of tissues in the path of the current may change due to the changing distance between the electrode and the phrenic nerve. Thus, any relative movement between the electrode and the phrenic nerve could affect the current applied to the phrenic nerve and surrounding non-targeted structures. Also, movement of the electrode relative to the phrenic nerve could cause damage to the surrounding vessels and tissue inside the patient's neck, cause pain and discomfort and reduce range of motion. In addition, wires and electrodes made of polymers and metals tend to fatigue and fracture when bent and twisted a lot. All these challenges are well known in the field of cervical phrenic nerve stimulation.

Electrode “cuffs” and tubular leads have been used to stimulate the phrenic nerve, such as shown in U.S. Pat. No. 10,596,368. Leads are essentially insulated electric wires with exposed electrodes. Electrode “cuffs” clamp or wrap around (or partially around) the nerve to ensure positive fixation and minimal current dispersion.

Electrode assemblies for stimulating nerves include placing flexible leads (rods with circumferential ring electrodes) adjacent the nerve. See Taira T, Takeda N, Itoh K, Oikawa A, Hori T. Phrenic nerve stimulation for diaphragm pacing with a spinal cord stimulator: technical note. Surg Neurol. 2003 Feb.;59(2): 128-32; discussion 132. doi: 10.1016/s0090-3019 (02) 00997-7. PMID: 12648917 and U.S. Patent Application Publication 2019/0269911. The rods with ring electrodes may not be secured to tissue to allow the rod to be removed or may be secured with sutures that require a relatively large opening in the neck to allow a surgeon to place the sutures. Electrodes for stimulating a nerve may be arranged on a panel as shown in U.S. Patent Application Publication 2014/0358026. A percutaneous approach for implanting an electrode in the neck of a patient is shown in U.S. Pat. No. 9,486,628.

A risk of implanting a nerve stimulation electrode assembly in the neck is that the neck has many nerves, blood vessels and muscles in close proximity to each other that are at risk of being dissected or damaged during surgery. Also, surgery of the neck may cause painful muscle damage and loss of mobility as the surgeon cuts through muscle to move the electrode assembly into position around or under the peripheral nerve. Also, suture-secured electrodes can still move back and forth relative to the nerve when the patient bends or turns their neck.

In view of the risks and difficulties with surgically placing an electrode around or adjacent to a phrenic nerve, especially in the neck where it is relatively deep under tissue and muscle layers and not easily accessible, there are needs for an electrode assembly that is specifically designed to accommodate the cervical phrenic nerve anatomy and a minimally invasive method of surgery to implant the electrode assembly near the phrenic nerve to treat sleep apnea.

### SUMMARY OF INVENTION

A minimally invasive surgical method has been invented to implant an electrode assembly through the neck (cervical approach) to stimulate the phrenic nerve sufficiently to move the diaphragm in a safe and controllable manner and to treat sleep apnea. The surgical method takes advantage of the natural neck anatomy and may be performed as an outpatient procedure. The procedure may be performed with the patient under a general anesthesia, conscious sedation and/or under a local anesthesia, such as using a subcutaneous (SC) administered analgesic drug.

One target site for an incision is located in or near the posterior triangle of the neck on the right, left or both sides during one procedure. This target site is over the phrenic nerve and spaced from major blood vessels and other important nerves. Diagnostic ultrasound imaging may be used to locate a portion of the phrenic nerve distant from major blood vessels and other nerves, such as the brachial plexus.

The surgical procedure may include a small, for example 2 to 5 cm, incision in the skin and through tissue. The location of the incision is approximately 2 cm above and parallel to the mid-portion of the clavicle. Alternatively, the incision may be performed above the clavicle in an oblique or perpendicular direction depending on patient-specific characteristics, such as subcutaneous fat deposition and scar tissue. Through the incision, the thin superficial platysma muscle is divided. The sternocleidomastoideole is dissected from adjacent tissues but not cut, and is reflected medially to expose the prescalene fat pad which is moved laterally to expose the prevertebral fascia of the deep cervical fascia.

The phrenic nerve is a bundle of fibers enclosed in a sheath. The prevertebral fascia may be incised to expose the phrenic nerve sheath and the anterior surface of the anterior scalene muscle. Alternatively, the prevertebral fascia may not be incised. The phrenic nerve sheath need not be resected. It should be understood that any reference herein to a nerve includes the nerve fiber bundle and the sheath.

A fascia is a thin sheet of connective tissue, which is primarily collagen, and functions to stabilize, enclose, and separates muscles and other internal organs. The prevertebral fascia is continuous with the transversalis fascia of the thorax and abdomen. The prevertebral fascia forms a natural boundary between muscles in the neck behind the trachea. It is a transparent and durable membrane, and the nerve can be visualized through it under normal or enhanced light, such as polarized or monochromatic light.

The anterior surface of the anterior scalene muscle includes the epimysium which is an external sheath surrounding the anterior scalene muscle. The phrenic nerve is on or at least partially embedded in the anterior surface of the anterior scalene muscle and is between the prevertebral fascia and the anterior surface of the anterior scalene muscle.

A nerve test probe may be used to identify and locate the phrenic nerve. This probe can be part of the system described below. There is no need to separate the phrenic nerve from the anterior scalene muscle or to tunnel between the nerve and the anterior scalene muscle. Further, it may not be necessary to incise the prevertebral fascia to expose the underlying phrenic nerve and the muscle since the fascia is thin and transparent.

Once the phrenic nerve is located on the anterior scalene muscle, an electrode assembly, which may include bipolar or tripolar paddle electrodes, is placed over the phrenic nerve. The electrode assembly may be anchored to the anterior scalene muscle and/or the prevertebral fascia of the deep cervical fascia, such as with sutures; micro needles, micro-hooks, micro-teeth, a micro-patterned dry adhesive and/or adhesive. These attachment aids can be biodegradable and eventually bio-absorbed into the body since the assembly in time will be held in place by fibrosis of tissues.

The electrode assembly may be positioned over the phrenic nerve, between the anterior surface of the anterior scalene muscle and the prevertebral fascia of the deep cervical fascia (prevertebral fascia). Alternatively, the electrode assembly may be positioned over the phrenic nerve and the fascia, anchored to the anterior surface of the prevertebral fascia and between the anterior surface of the prevertebral fascia and the posterior surface of the sternocleidomastoideole.

At least one outer surface, e.g., the anterior surface, of the electrode assembly may have a low friction coating, e.g., PTFE (polytetrafluoroethylene). The low friction lubricious coating allows the surface of the electrode assembly to discourage ingrowth and slide with respect to the sternocleidomastoideole or the prevertebral fascia while the electrode assembly remains anchored to the prevertebral fascia or the anterior scalene muscle. Conversely, the surface facing the nerve may have a porosity of material known to encourage ingrowth to facilitate immobilization of the electrodes with respect to the phrenic nerve.

The electrode assembly is in electrical communication, such as by a wire lead or flex circuit, to a controller such as an implantable pulse generator (IPG) or an external pulse generator (EPG) equipped with an RF link to transfer energy to the subcutaneous antenna, which can be a passive or active antenna. The pulse generator provides the electrical signals transmitted to the electrode assembly and applied by electrodes in the assembly to stimulate the phrenic nerve. For an IPG, the pulse generator may be surgically implanted in the subclavian pocket, e.g., the infraclavicular region, and a wire lead may be surgically implanted by tunneling through tissue, e.g. the prescalene fat pad, to extend from the implanted pulse generator to the electrode assembly.

The pulse generator may house a battery and include an antenna to communicate to and receive power from the devices external to the body of the patient. The devices may include a recharger that recharges the battery and a processor configured to receive data from the pulse generator and to transmit, for example, updates to algorithms for stimulating the phrenic nerve. To facilitate communications with and recharging of the pulse generator a patch on the skin or another wearable device may include antennas and other electronics configured to relay power and communications between the pulse generator and an external device(s).

The pulse generator may be integrated with or be in communication, e.g., wired or wirelessly, with other devices, such as devices sensing respiration of the patient and body position movements of the patient. Data from these other devices may be used by the pulse generator to determine when and whether or how much to stimulate the phrenic nerve to induce the diaphragm to contract or to contract further than would occur without the stimulation. The sensors integrated into the device can be bioimpedance sensing, accelerometry, and acoustic microphones to monitor breathing.

The invention may be embodied as a method for surgically implanting a device in a living human patient to treat sleep apnea, the method comprising: surgically positioning an electrode assembly over a portion of a phrenic nerve and over an anterior surface of an anterior scalene muscle, wherein the positioning does not separate the phrenic nerve from the anterior surface of an anterior scalene muscle, and affixing the electrode assembly to the anterior surface or a prevertebral fascia covering the anterior surface.

The invention may be embodied as a system for the treatment of sleep apnea in a patient, the system comprising: a plate or paddle electrode assembly comprising a flexible panel and at least one pair of conductive stimulation electrodes disposed on a first surface of the panel; and a neurostimulation device configured to transfer stimulatory electric current to the at least one pair of stimulation electrodes; wherein the plate or paddle electrode assembly is configured to be affixed to tissue proximate to an anterior scalene muscle with the first surface of the electrode assembly abutting the anterior scalene muscle or a prevertebral fascia covering an anterior surface of the anterior scalene muscle, and wherein the at least one pair of stimulation electrodes is configured to deliver current to activate the phrenic nerve through application of an electric field to a section of the phrenic nerve.

The invention may be embodied as a system for the treatment of sleep apnea in a patient by stimulation of the phrenic nerve during inspiration and expiration periods at a set breathing rate where both periods of stimulation forced diaphragm contraction in excess of unstimulated state and where the inspiration period energy is higher than expiration period energy.

The invention may be embodied as a delivery tool configured to position an electrode assembly in a patient's neck, the delivery tool comprising: an electrode carrying portion including a receiving space with at least one aperture in a floor of the receiving space, the receiving space is configured to hold the electrode assembly; and a lead carrying portion extending from the electrode carrying portion, the lead carrying portion configured to hold a lead connected or connectable to the electrode assembly.

The receiving space may be recessed and configured to releasably hold the electrode assembly. The at least one aperture may comprise a pair of through apertures on the floor of the receiving space. The receiving space may be bound by a rim. The rim may emerge from a perimeter of the floor of the receiving space and surrounds a major portion of said floor, optionally wherein the rim is a continuous rim surrounding the floor and having opposite ends joining the lead portion.

The floor perimeter may be shaped such that an electrode assembly received in the receiving space and provided with a perimeter in part or entirely complementarily shaped to that of the floor cannot rotate and/or shift relative to the electrode carrying portion. The floor perimeter may not be circular and/or comprise one or more lobes. The floor of the receiving space may be provided with means configured for releasably connecting the electrode assembly to the electrode carrying portion.

The means configured for releasably connecting the electrode assembly to the electrode carrying portion may comprise one or more of: an adhesive coating, a hook and loop connection, a clip.

The lead carrier may have an elongated shape, optionally with a length of the lead carrying portion being at least 10 times a width of the same lead carrying portion, and wherein the electrode carrying portion has a paddle-like shape with a maximum width which is larger, optionally at least two times larger, than the width of the lead carrying portion.

The electrode carrying portion may be configured to pivot relative to the lead carrier. The electrode carrying portion may be configured to flex relative to the lead carrier or wherein the delivery tool has a hinge, optionally a living hinge, at a transition point between the electrode carrying portion and the lead carrier.

The lead carrying portion may comprise a channel configured to hold the lead connected or connectable to the electrode assembly. The receiving space may transition to the channel. The channel may extend the length of the lead carrier, optionally wherein the channel extends parallel to the central longitudinal axis (a) of the delivery tool.

The delivery tool may include a visual alignment feature configured to aid in the alignment of the electrode assembly with a stimulation target. The visual alignment feature may be at the transition point. The visual nerve alignment feature may comprise a marking or a raised features or a notch or an indentation providing a visual aid for aligning the electrode assembly.

The electrode carrying portion may be transparent or wherein the lead carrying portion is transparent or wherein both the electrode carrying portion and the lead carrying portion are transparent. The electrode carrying portion may be integrally formed with the lead carrying portion.

The delivery tool may include a handle connected at an end of the lead carrying portion opposite to the electrode carrying portion. The channel may extend at least part way into the handle. The handle may be more rigid than the lead carrying portion and/or more rigid than the electrode carrying portion.

The delivery tool may further comprise a securing device configured to hold the electrode assembly in place relative to a stimulation target when the delivery tool is inserted into a patient's body, the securing device being further configured to allow the electrode to be repositioned after the electrode assembly has been held in place by the securing device. The securing device may comprises a pair protrusions, the pair of protrusions being spaced apart from each other on opposite zones of the same side of the electrode carrying portion. The pair of protrusions may extend from the rim of the receiving space or extend from a portion of the floor adjacent to the rim, optionally wherein the securing device comprises protrusions emerging from the lead carrying portion. Each one of the protrusions may end with a rounded engagement surface so that the protrusions allow the electrode assembly carrier to be repositioned without puncturing underlying patient's tissue. The protrusions may project from central zone or from a proximal zone of the electrode assembly carrier.

A distal end of the delivery tool may include an integrated neuromonitoring nerve detecting tip configured for emitting a signal indicating close electric contact between the detecting tip and a patient's neuronal structure.

The delivery tool of may include a cable segment integrated in the delivery tool and comprising a first connector for connection with the electrode assembly and a second connector for direct connection with an electric current generator connection or with a further cable segment connectable to the electric current generator, wherein the current generator is either integrated in the delivery tool or a device external to the delivery tool. The cable segment may be integrated in the lead carrying portion and defines the lead connected or connectable to the electrode assembly. The electric current generator may be integrated into the delivery tool, optionally housed in the/a handle of the delivery tool. The electric current generator may comprise a power source, optionally including at least one battery, and electronic circuitry configured to deliver electrical current through said lead to the electrode assembly mounted on a distal end of the delivery tool.

The delivery tool may further comprise a user interface, optionally including a touch sensitive item on the handle, communicatively connected to the electronic circuitry and configured to be operated by a user to issue a command for the electronic circuitry, wherein the electronic circuit is configured to receive said command and upon receipt of said command control the electric current generator to deliver electrical current to the electrode assembly to stimulate the phrenic nerve during the placement and/or securement of the electrode assembly. The lead may be configured to be detached from the delivery tool and electrically coupled to a pulse generator that may be implanted in the patient or positioned next to the patient. The delivery tool may be disposable. The delivery tool may be packaged in a sterile kit ready to be used by a physician.

The invention may be embodied as an electrode assembly for implantation in a patient's neck comprising: a panel portion with a pair of apertures and a pair of electrodes, and a lead portion extending from the panel portion, the lead portion comprising at least one wire configured to convey a stimulation current between the electrodes and a current source.

The pair of electrodes may be located between the pair of apertures. The pair of electrodes and the pair of apertures may be coplanar. The panel portion may be a paddle-shaped panel portion. The panel portion may be in the form of a wide and flat membrane.

The electrodes may be in the form of a conductive strip, optionally wherein the electrodes are unilateral bipolar electrodes in the form of conductive strips.

The pair of apertures may be located at opposite ends of the panel portion, while the pair of electrodes are located at an intermediate zone of the panel portion so that the pair electrodes are located between the pair apertures.

The panel portion may include conductive leads positioned on, or embedded within, the panel portion and configured to connect the pair of electrodes to the at least one wire inside the lead portion. The lead portion may be long and thin. The lead portion may include a proximal end with at least one electrical contact, in particular a pair of electrical contacts, configured to connect to a pulse generator or to a lead extension. The lead portion may comprise a strain relief device configured to anchor the lead portion to the patient's muscle or connective tissue. The strain relief device may be an auxiliary aperture.

The electrode assembly may be configured to be anchored to the patient's anterior scalene muscle at the apertures in the panel portion. The apertures in the panel portion may be configured to receive fasteners.

The panel portion may be formed from a flexible, transparent material. The lead portion may be formed from a flexible, transparent material. The electrodes may be embedded in the flexible, transparent material of the panel portion. The panel portion may be made from dielectric material, optionally from biocompatible dielectric material, such as PI, wherein the electrodes are electrically isolated from each other. The panel portion may or may not be circular and/or comprises one or more lobes. The panel portion may include one or more impedance sensors positioned on or embedded within said panel portion, in particular wherein the impedance sensors include tracheal flow impedance sensing leads that are positioned to sense a volume of air inside the patient's airways and lungs.

The electrodes may be separated by a gap in a range of 1 mm to 10 mm that extends the length of the strips forming the electrodes. The at least one pair of electrodes may be configured to generate an electric field having electric field lines that run partially or substantially parallel to nerve fibers of a targeted section of the phrenic nerve, when the electrode assembly is in position above the phrenic nerve.

The panel portion may have a front side and a back side and wherein the electrode assembly has a fibrosing surface on at least one of the front and back sides of the panel portion, wherein fibrosing surface is configured to promotes fibrotic tissue formation to anchor the electrode assembly to underlying tissue.

The fibrosing surface of the electrode assembly may include one or more of a layer of porous material that encourages ingrowth of tissue, a medical adhesive that adheres to muscle, micro-needles, micro-gripping hydrophobic patterns or gecko feet pattern. The fibrosing surface of the electrode assembly may be confined to the back side of the panel portion of the electrode assembly.

The front side of the panel portion may comprise an anti-fibrotic surface.

The electrode assembly may include a low friction, non-adhesive, smooth coating or layer forming the anti-fibrotic surface, optionally wherein the smooth coating or layer is formed from silicone, hydrogel, PTFE (polytetrafluoroethylene) or a hydrophilic coating.

The invention may be embodied as a system for the treatment of sleep apnea in a patient, the system comprising: the electrode assembly described above, a neurostimulation device configured to transfer stimulatory electric current to the at least one pair of stimulation electrodes; wherein the plate or paddle electrode assembly is configured to be affixed to an anterior scalene muscle with the first surface of the electrode assembly abutting the anterior scalene muscle or a prevertebral fascia covering an anterior surface of the anterior scalene muscle, and wherein the at least one pair of stimulation electrodes is configured to deliver current to activate the phrenic nerve through an application of an electric field to a section of the phrenic nerve.

The system may further comprise a delivery tool. The electrode assembly may be removably mounted on the delivery tool. The panel portion of the electrode assembly may be held in place in the receiving space of the electrode carrying portion in a removable manner. The perimeter of the receiving space or of the floor of the receiving space may have the same shape as a perimeter or a portion of the perimeter of the panel portion of the electrode assembly.

The receiving space may be the same as or slightly larger than the size of the panel portion so that the panel portion fits snuggly in the receiving space. The panel portion may be held in place in the receiving space in a removable manner by friction or adhesively or by a hook and loop fastener. The shape of the perimeter of the receiving space may prevent the panel portion from rotating and/or shifting while in the receiving space.

The apertures positioned within the floor of the receiving space may align with apertures in the panel portion of the electrode assembly when the panel portion is positioned within the receiving space, in particular wherein the alignment between the apertures and the apertures allows for the electrode assembly to be permanently secured to the anterior scalene muscle while the electrode assembly is being held in place by the delivery tool. The apertures in the floor of the receiving space are larger than the apertures in the panel portion of the electrode assembly.

The delivery tool may have a channel with a width and depth marginally larger than a diameter or width of the lead portion so that the channel can accommodate said lead portion. The e channel may be sized to allow the lead portion to slide along the length of the channel so that, when the delivery tool is removed from the lead portion, the delivery tool does not pull on the lead portion or on the electrode assembly.

A fixation tool configured to secure the electrode assembly in the patient's neck may be used with the deliver too. The fixation tool may be integrated into the delivery tool.

Also, a neurostimulation device may be used with the delivery tool during the implantation of the electrode. The neurostimulation device may comprise a pulse generator, which is either an implantable pulse generator (IPG) or an external pulse generator (EPG), wherein the pulse generator, in particular including a programmable or programmed controller, is configured to generate and transmit to the electrodes of electrode assembly electrical signals applied by electrodes to stimulate the phrenic nerve. The pulse generator may include a battery and an antenna to communicate to and receive power from devices external to the body of the patient. The pulse generator may be configured to communicatively receive from devices external to the body of the patient updates to algorithms executable by the pulse generator for stimulating the phrenic nerve.

The pulse generator may comprise or is in communication with an additional device comprising one or more of a device for sensing respiration of the patient and a device for sensing body position movements of the patient, and wherein the pulse generator is configured for receiving data from the additional device and use the data from the additional device to determine when, or whether, or how much to simulate the phrenic nerve to induce the diaphragm to contract or to contract further than would occur without the stimulation. The pulse generator may comprise an external pulse generator (EPG), and wherein a lead extension bridges a distance between the electrode assembly and the external current generator one end of the lead extension being connected to the external current generator, while the other end of the lead extension being connected to the wire or lead of the electrode assembly.

The pulse generator, such as an external pulse generator, may be configured to generate a test stimulation current for determining correct positioning of the electrode assembly. The test stimulation current may comprise ramping up the test stimulation current maintaining the test stimulation current within a range of 1.0 to 5.0 mA.

The neuromodulation device, in particular the external pulse generator, may be configured to generate after generation of the test stimulation current, optionally after the delivery tool and the electrode assembly have been positioned at an elected location, a further current higher than the test stimulation current to be applied to the phrenic nerve, optionally wherein the further current is at 5 mA.

The invention may be embodied as a method to surgically implanting an electrode assembly in a living human patient to treat sleep apnea, the method comprising: making an incision on or adjacent to the patient's neck; selecting a path from the incision to a portion of one of the patient's phrenic nerves lying in the patient's subclavian triangle, the path following the natural boundaries of muscles and blood vessels formed by fascia and fat pads; opening the incision along the selected path to create a surgical opening that exposes said portion of the patient's phrenic nerve at the patient's subclavian triangle without cutting the patient's muscle tissue and blood vessels; inserting a delivery tool that carries an electrode assembly into the surgical opening; maneuvering the delivery tool to position and reposition the electrode assembly on an anterior surface of the patient's anterior scalene muscle and on said portion of the patient's phrenic nerve without separating said phrenic nerve from the anterior surface of the anterior scalene muscle; applying a stimulation current to the phrenic nerve and detecting the patient's response to the stimulation current after each time the electrode is positioned on the patient's anterior scalene muscle and phrenic nerve; selecting a final position for the phrenic nerve based on the patient's response to the applications of the stimulation current; securing the electrode assembly to the anterior surface of the patient's anterior scalene muscle at the selected final position; and separating the delivery tool from the electrode assembly and removing the delivery tool from the surgical opening after the electrode assembly has been secured to the anterior surface of the patient's anterior scalene muscle at the selected final position.

The invention may be embodied as a delivery tool configured to surgically position an electrode assembly on a patient's anterior scalene muscle and phrenic nerve, the delivery tool comprising: an electrode carrying portion comprising a receiving space with at least one aperture in a floor of the receiving space, the receiving space being configured to hold an electrode assembly; a lead carrying portion extending from the electrode carrying portion, the lead carrying portion being configured to hold a lead connected to the electrode assembly; and a securing device configured to hold the electrode assembly in place relative to a stimulation target when the delivery tool is inserted into a patient's body, the securing device being further configured to allow the electrode to be repositioned after the electrode assembly has been held in place by the securing device, wherein the electrode carrying portion and the lead carrying portion are transparent.

The invention may be embodied as an electrode assembly configured to be positioned on a patient's anterior scalene muscle and phrenic nerve, the electrode assembly comprising: a panel portion with a pair of apertures and a pair of electrodes between the pair of apertures, the pair of electrodes and the pair of apertures being coplanar; and a lead portion extending from the panel portion, the lead portion comprising at least one wire configured to convey a stimulation current between the electrodes and a current source, wherein the panel portion and the lead portion are formed from a flexible, transparent material.

The invention may be embodied as a system for the treatment of sleep apnea in a patient, the system comprising: a plate or paddle electrode assembly comprising a flexible panel and at least one pair of conductive stimulation electrodes disposed on a first surface of the panel; and a neurostimulation device configured to transfer stimulatory electric current to the at least one pair of stimulation electrodes; wherein the plate or paddle electrode assembly is configured to be affixed to an anterior scalene muscle with the first surface of the electrode assembly abutting the anterior scalene muscle or a prevertebral fascia covering an anterior surface of the anterior scalene muscle, and wherein the at least one pair of stimulation electrodes is configured to deliver current to activate the phrenic nerve through an application of an electric field to a section of the phrenic nerve.

The system may comprise a delivery tool configured to position the electrode assembly on the anterior scalene muscle. The delivery tool may be configured to hold the electrode assembly in position on the anterior scalene and reposition the electrode assembly. The delivery tool may be configured to temporarily secure the electrode assembly at multiple positions on the anterior scalene muscle and reposition the electrode assembly between the multiple positions.

The delivery tool may have a distal end with an electrode carrier configured to hold electrode assembly while the electrode assembly is position over the phrenic nerve and fastened to the anterior scalene muscle. The delivery tool may comprise a first aperture and the electrode assembly comprises a second aperture, and wherein the first aperture is aligned with the second aperture while the delivery tool holds the electrode assembly so that the first and second apertures are configured to receive the same fastener at the same time.

The delivery tool, the plate or paddle electrode assembly and the neurostimulation device may be preassembled and housed in a package.

The flexible panel may be a dielectric, and the conductive stimulation electrodes may be electrically isolated from each other.

The at least one pair of stimulation electrodes may be in a flex circuit.

The invention may be embodied as a delivery tool configured to position an electrode assembly in a patient's neck, the delivery tool comprising: an electrode carrying portion including a receiving space with at least one aperture in a floor of the receiving space, the receiving space is configured to hold the electrode assembly; a lead carrying portion extending from the electrode carrying portion, the lead carrying portion configured to hold a lead connected or connectable to the electrode assembly; an electric current generator with an electrical connector that is configured to be connected to a lead with electrodes; and a grip portion configured to be gripped by a user, wherein the delivery tool is configured to be held by the user when the user at least at the grip portion.

The delivery tool may include a user interface configured to receive an input from the user to selectively actuate and disable the electric current generator. The user interface includes at least one of a button, a dial, a trigger, and a lever. The user interface may include a microphone and the electric current generator is voice activated. The user interface may be located in or on the grip portion.

The delivery tool may include an indicator configured to provide a signal to the user representing a condition and/or position of an electrode positioned on the electrode carrying portion. The indicator may include at least one of a light, an audible speaker, and a vibrator. The signal may be representative of an amount of power applied to the electrode. indicate to the physician that electrical power is being applied to the electrode and/or whether the electrical current delivered by the electrodes is stimulating the phrenic nerve. The indicator may be located on or in the grip portion.

The electric current generator may be positioned within the grip portion. The lead carrying portion may be configured to convey the electric current generated by the electric current generator to the electrode held by the electrode carrying portion.

The receiving space may be recessed and configured to releasably hold the electrode assembly. The at least one aperture may comprise a pair of through apertures on the floor of the receiving space. The receiving space is bound by a rim. The receiving space, in particular the floor of the receiving space, may be provided with means configured for releasably connecting the electrode assembly to the electrode carrying portion.

The means configured for releasably connecting the electrode assembly to the electrode carrying portion comprises one or more of: an adhesive coating, a hook and loop connection, a clip. The electrode carrying portion may be configured to pivot relative to the lead carrier.

The electrode carrying portion may be configured to flex relative to the lead carrier or wherein the delivery tool has a hinge, optionally a living hinge, at a transition point between the electrode carrying portion and the lead carrier.

The delivery tool may include a visual alignment feature configured to aid in the alignment of the electrode assembly with a stimulation target, wherein the visual nerve alignment feature comprises a marking or a raised features or a notch or an indentation providing a visual aid for aligning the electrode assembly.

The electrode carrying portion may be transparent or wherein the lead carrying portion is transparent or wherein both the electrode carrying portion and the lead carrying portion are transparent.

The delivery tool may further comprise a securing device configured to hold the electrode assembly in place relative to a stimulation target when the delivery tool is inserted into a patient's body, the securing device being further configured to allow the electrode to be repositioned after the electrode assembly has been held in place by the securing device.

The securing device may comprise a pair protrusions, the pair of protrusions being spaced apart from each other on opposite zones of a same side of the electrode carrying portion. The pair of protrusions may extend from the rim of the receiving space or extend from a portion of the floor adjacent to the rim, optionally wherein the securing device comprises protrusions emerging from the lead carrying portion.

A distal end of the delivery tool may include an integrated neuromonitoring nerve detecting tip configured for emitting a signal indicating close electric contact between the detecting tip and a patient's neuronal structure.

The invention may be embodied as a kit comprising the delivery tool according to any one of claims 149 to 173; and a sterile container that contains the delivery tool. The delivery tool may be sterile while in the package. The kit may include a fixation tool configured to secure the electrode in place.

### SUMMARY OF FIGURES

The figures in this application illustrate an exemplary surgical approach to implanting an electrode assembly to treat sleep apnea, the figures are:

FIG. 1 illustrates a side view of a patient prepared for surgery, a surgical site on a patient's neck, and the components of the phrenic nerve stimulation system.

FIG. 2 is another side view of a patient with the internal components of the phrenic nerve stimulation system implanted in the patient's neck.

FIGS. 3 to 4 are cut-away views of a patient's neck and show the internal anatomy of the neck.

FIG. 5 is a flow chart illustrating a method for implanting an electrode on the phrenic nerve.

FIGS. 6 and 7 show incision site locations on the neck of a patient.

FIG. 8 is a cross-sectional view of the neck to show the dimensions of the surgical opening once the patient's tissues have been retracted.

FIGS. 9 to 14 are cross-sectional views of the neck to show a surgical tool accessing the phrenic nerve to implant an electrode assembly over the nerve.

FIG. 15 is a cross-sectional view of the neck showing the electrode assembly implanted over the phrenic nerve and the incision site closed.

FIGS. 16A to 16D are cut-away views of the patient showing strain relief locations for the electrode lead.

FIG. 17 is a cross-sectional view of the neck to showing a second embodiment of the surgical tool inserting through an incision in the next and accessing the anterior scalene muscle to implant an electrode assembly over the phrenic nerve.

FIG. 18 is an illustration of a therapy waveform.

FIG. 19 is a close-up perspective view of an electrode assembly over a phrenic nerve and attached to an anterior surface of the anterior scalene muscle.

FIG. 20A is a perspective view of an embodiment of a di-pole electrode panel assembly overlying the phrenic nerve.

FIG. 20B is an end view of the electrode panel assembly and a cross-sectional view of the phrenic nerve.

FIGS. 21A and 21B are a perspective view and an end view, respectively, of another di-pole electrode panel assembly mounted over a phrenic nerve.

FIGS. 22A and 22B are a perspective view and an end view, respectively, of a tri-pole electrode panel assembly mounted over a phrenic nerve.

FIGS. 23-26 are perspective views of exemplary electrode panel assemblies.

FIGS. 27A and 27B show another exemplary electrode panel assembly.

FIGS. 28A and 28B show another embodiment of a panel electrode assembly.

FIGS. 29A to 29B illustrate an exemplary delivery tool.

FIGS. 30A to 30B illustrate the electrode panel carrier on a distal end of a delivery tool.

FIG. 31 illustrates an electrode panel assembly seated in an electrode carrier portion of a delivery tool.

FIGS. 32A to 32C illustrate another embodiment of a panel electrode assembly.

### DETAILED DESCRIPTION

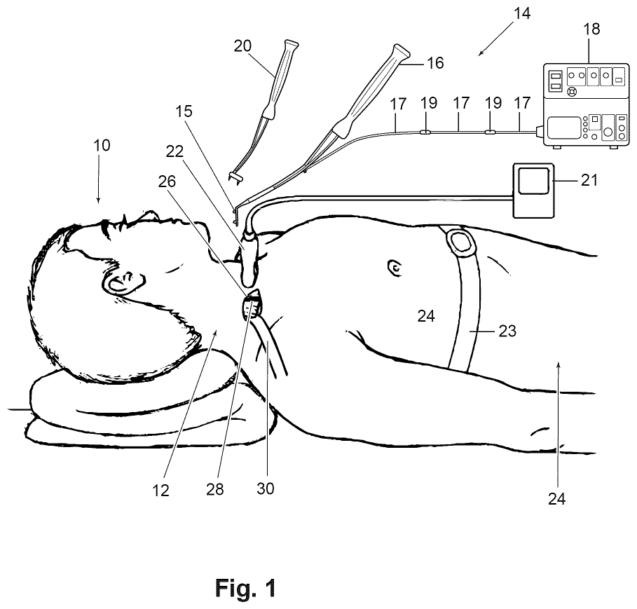

FIG. 1 shows a patient 10 prepared for a surgical procedure to implant an electrode assembly in the patient's neck 12. FIG. 1 also shows a surgical implantation/surgical implantation system 14 that is used to position, test, attach, and implant the electrode assembly in the patient's neck. The surgical implantation system 14 may include an electrode or electrode assembly 15 (e.g., single use electrode assembly) mounted on a delivery tool 16. The electrode assembly 15 may be connected by a cable extension 17 (e.g., a wire or lead) to an external current generator 18 that can be within or outside of the surgical field. In addition, it is contemplated that the delivery tool 16 may be disposable. The cable extension 17 may be segmented and include connectors 19 between segments. The particular segment that is directly attached to the delivery tool 16 may be integrated into the tool 16 such that the tool 16 and the cable extension 17 are provided in a sterile kit housed in the same package. The sterile segment of the cable extension may connect to another segment of the cable extension 17 that is non-sterile and may connect to the non-sterile external current generator.

Alternatively, the current generator 18 may be integrated into the delivery tool 16. The handle of the delivery tool 16 may house a battery and electronic circuitry that delivers electrical current through a conductive lead 41 to an implantable electrode 15 mounted on a distal end 98 of the delivery tool 16. A button 408 on the handle may be pushed by the physician to deliver electrical current to the electrode 15 to stimulate the phrenic nerve 28 during the placement and securement of the electrode 15. After the electrode 15 is properly placed over the phrenic nerve 28 and secured to the anterior scalene muscle 66, the lead 41 may be detached from the delivery tool 16 and electrically coupled to a pulse generator 38 that may be implanted in the patient 10 or positioned next to the patient's throat while the patient 10 sleeps. The integrated delivery tool 16 and current generator 18 may be packaged in a sterile kit ready to be used by a physician. The delivery tool 16 and current generator 18 may be discarded after the electrode 15 is implanted. An advantage provided by an integrated delivery tool 16 and current generator 18 is that it becomes unnecessary to connect a cable extension to an unsterile external current generator.

The surgical implantation system 14 may also include a fixation tool 20 to secure the electrode assembly 15 in the patient's neck 12 as well as an ultrasound device 21 with a probe 22. In addition, a respiratory belt 23 may be mounted to the patient's abdomen 24 to measure the patient's diaphragmatic contractions.

The patient 10 may be prepared for surgery by placing the patient 10 in a supine or lateral position, such as a left lateral position for right phrenic nerve access. The neck area is prepared with sterile disinfection and surgical draping. In addition, the patient 10 may be given a local anesthetic, sedation, or general anesthesia.

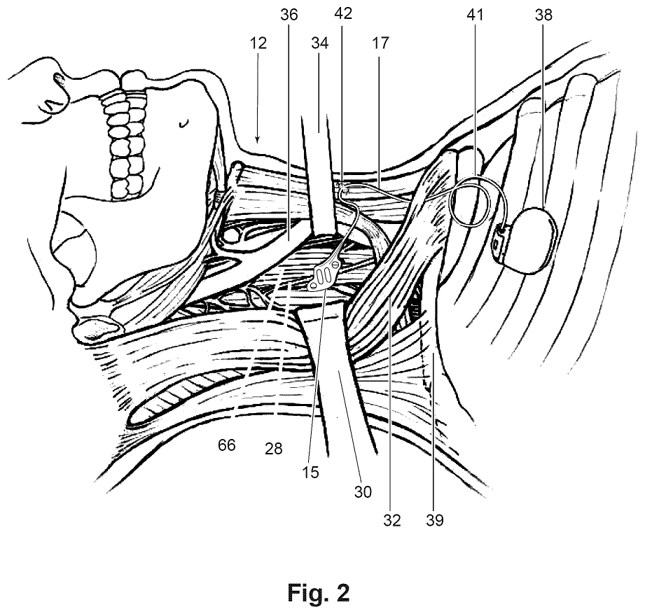

FIG. 1 illustrates a cervical incision site 26 that is suited to surgically form a passage through natural tissue interfaces (as described below) in the patient's neck 12 to access at least one of the phrenic nerves 28. A first retraction tool 30 may be positioned to retract the patient's sternocleidomastoideole 32 (see also FIG. 2). In addition, a second retraction tool 34 may be positioned to retract the patient's internal jugular vein 36 (see FIG. 2) so that the phrenic nerve 28 is exposed.

FIG. 2 illustrates the electrode assembly 15 that has been implanted in the patient's neck 12 adjacent to the phrenic nerve 28. As can be seen, in order to implant the electrode assembly 15, structures in the patient's neck 12 (such as the sternocleidomastoideole 32 and the internal jugular vein 36) are retracted and pulled back without cutting through any structures in order to provide access to the phrenic nerve 28. Once implanted in the patient's neck 12, the electrode assembly 15 may be connected to an implanted pulse generator (IPG) 38 by way of a wire or lead 41. The wire or lead 41 may include strain relief devices (strain relief anchors) 42 and all or part of the lead extension 17. There may be two or more strain relief devices 42 sutured to muscle or connective tissue to prevent the lead 41 from being pulled out of the IPG 38.

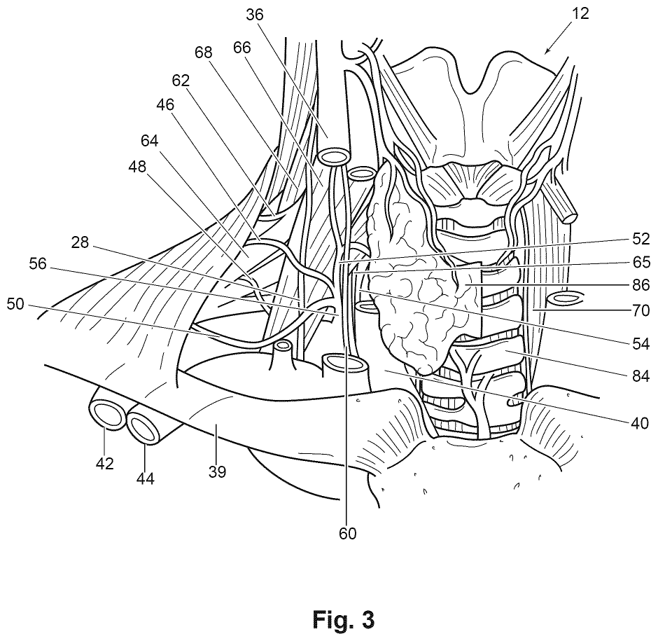

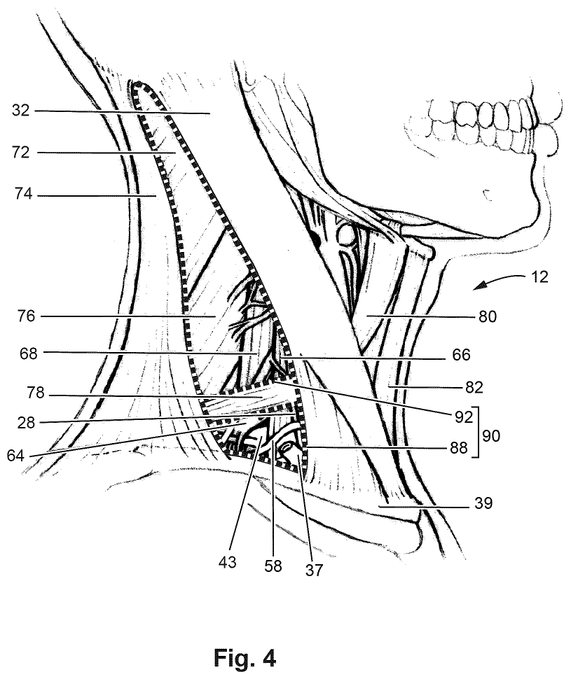

FIGS. 3 to 4 illustrate the internal anatomy in the patient's neck 12 just above the clavicle 39. The neck anatomy is dense with nerves, major blood vessels, muscles and other internal organs that make surgery of the neck difficult and risky. The patient's neck 12 includes major blood vessels, such as the internal jugular vein 36, the external jugular vein 37, the carotid artery 40, the subclavian artery 43, the subclavian vein 44, the transverse cervical artery 46, the dorsal scapular artery 48, suprascapular artery 50, the inferior thyroid artery 52, the vertebral artery 54, the thyrocervical artery 56, and the suprascapular artery 58. In addition to the phrenic nerve 28, the neck 12 contains the vagus nerve 60, the dorsal scapular nerve 62, the brachial plexus nerves 64, and the middle cervical sympathetic ganglion 65. Muscles in the neck 12 include the sternocleidomastoideole 32, the anterior scalene muscle 66, the middle scalene muscle 68, longus colli muscle 70, the splenius capitis muscle 72, the trapezius muscle 74, the levator scapulae muscle 76, the omohyoid muscle (inferior belly) 78, the omohyoid muscle (superior belly) 80, and the sternohyoid muscle 82. The neck 12 also includes the trachea 84 and the thyroid 86. The presence of these blood vessels, nerves and muscles in the neck 12 create difficulties and risks for a surgical approach into the neck 12 to access the phrenic nerve 28 and implant the electrode assembly 15 to stimulate the phrenic nerve 28.

Anatomic structures in the neck 12 which require particular attention when accessing the phrenic nerve 28 include the subclavian artery 43 and subclavian vein 44 on the inferior (lower) side of the phrenic nerve 28. On the anterior side of the phrenic nerve 28, the structures within the carotid sheath require particular attention. These structures include the internal jugular vein 36, the carotid artery 40, and the vagus nerve 60. Superior-laterally relative to the phrenic nerve 28, the brachial plexus nerves 64 require special attention. The brachial plexus nerves 64, derived from C5-C8 fibers, leave the neck region between the anterior and middle scalene muscles 66, 68. The internal jugular vein 36, the external jugular vein 37, and the transverse cervical artery 46 traverse the cervical incision site 26, and injury to these veins and arteries should be avoided to prevent bleeding.

The phrenic nerve 28 is located on an anterior surface of the anterior scalene muscle 66, while the brachial plexus nerves 64 are located on a posterior side of the anterior scalene muscle 66. Thus, the anterior scalene muscle 66 intervenes between the brachial plexus nerves 64 and the phrenic nerve 28. Accordingly, the location of the phrenic nerve 28 on the anterior scalene muscle 66 allows the phrenic nerve 28 to be accessed while safely avoiding the brachial plexus nerves 64, which is important because it is desired to avoid stimulating the plexus nerves 64 during apnea therapy.

The phrenic nerve 28 may be accessed through the patient's subclavian triangle 88 of the neck 12 which is the lower part of the patient's posterior triangle 90. The posterior triangle 90 is formed by the posterior border of the sternocleidomastoideole 32, the anterior border of the trapezius muscle 74 (both structures with their proximity to each other cranially creating the superior apex of the triangle) and the clavicle 39. The posterior triangle 90 of the neck 12 may be further subdivided based on separation through the omohyoid muscle 78. Thereby again forming two triangular regions, the inferior part (inferior to the omohyoid muscle 78) is referred to as the subclavian triangle 88 and the superior part called the occipital triangle 92.

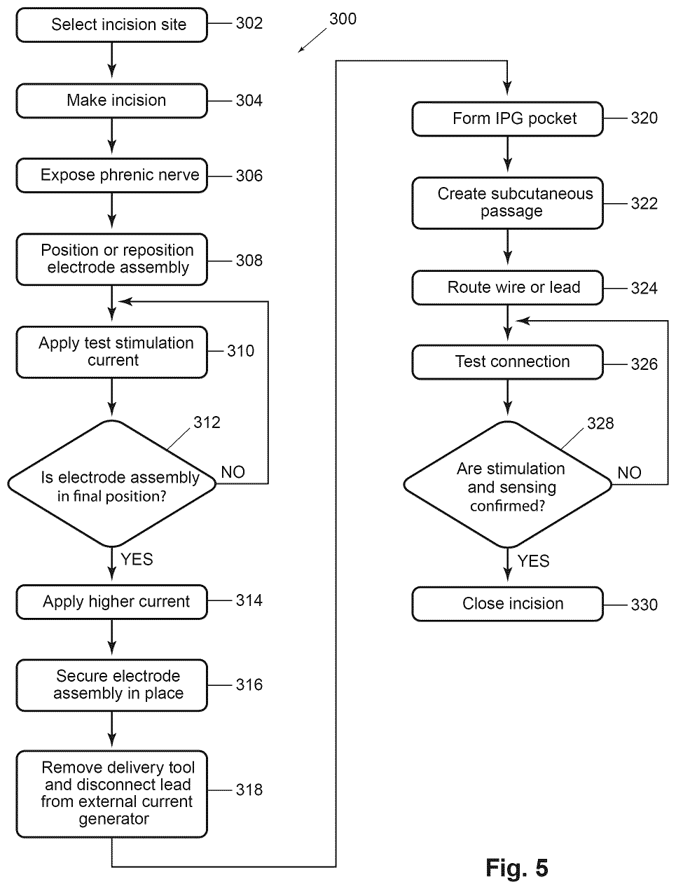

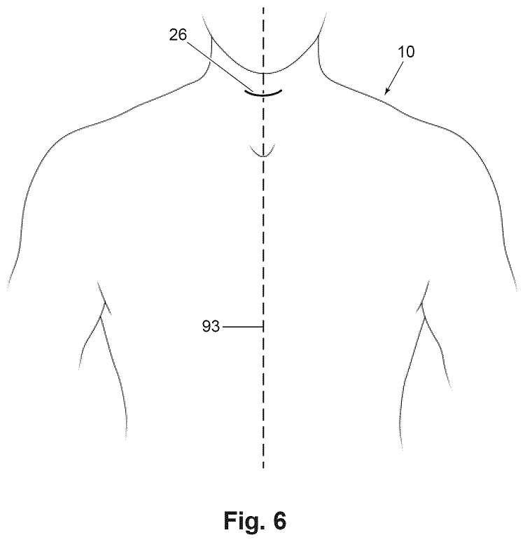

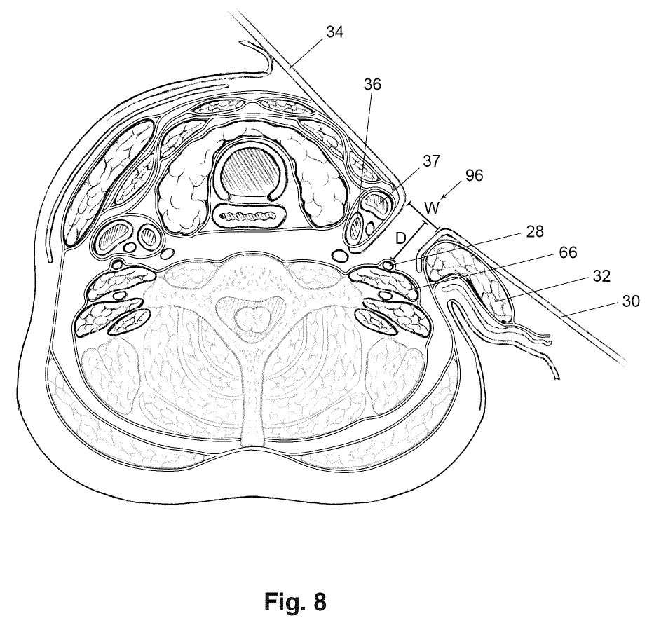

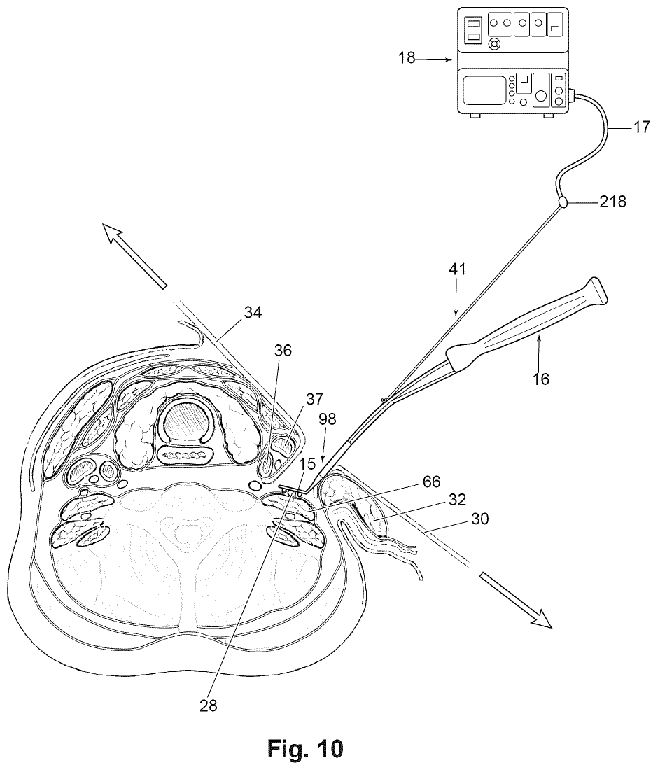

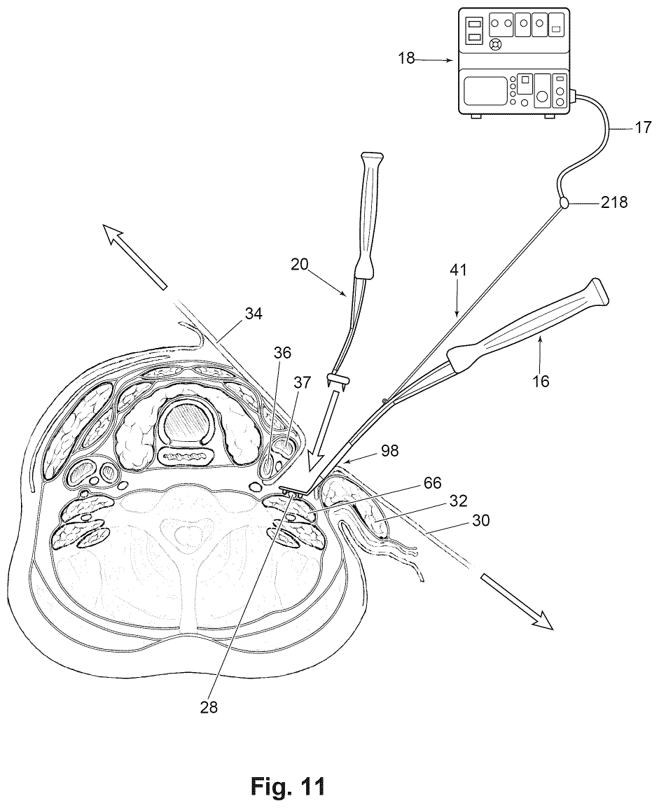

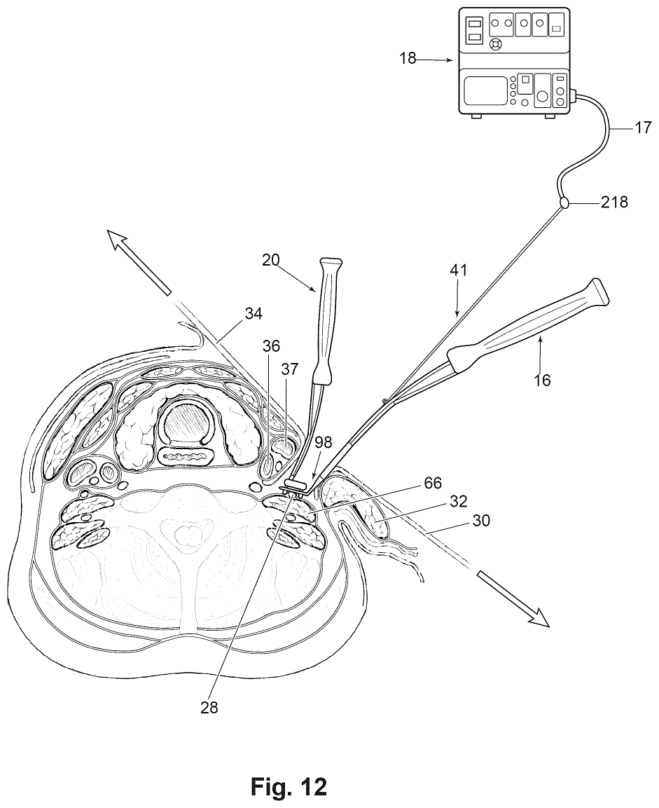

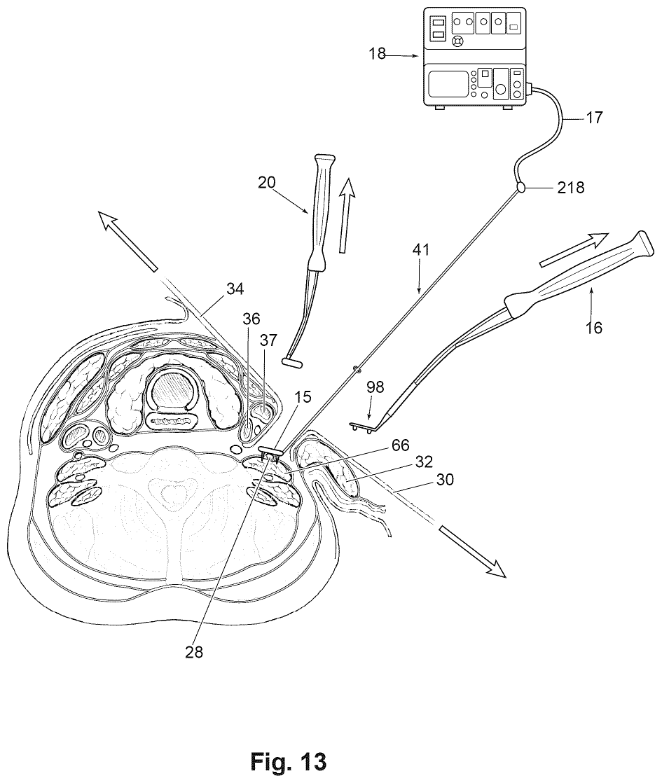

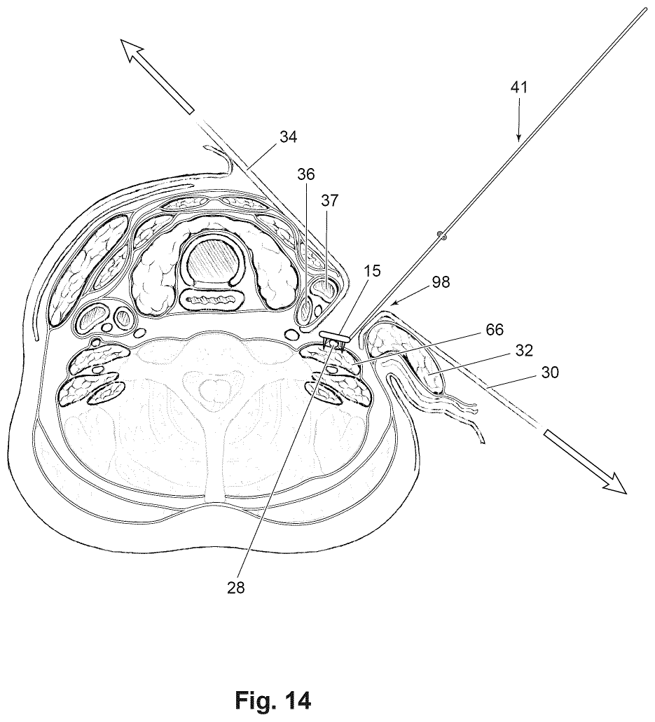

FIG. 5 is a flow chart describing an exemplary method 300 for implanting and securing the electrode assembly 15 in the patient's neck 12. FIGS. 6-14 illustrate how the method 300 is performed on the patient's neck 12.

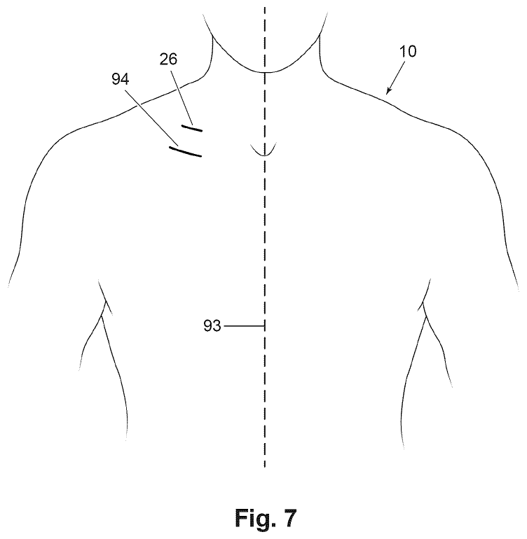

The method 300 may begin with step 302 in which the location of the cervical incision site 26 is selected. The location of the cervical incision site 26 may be selected based on how the phrenic nerve 28 is to be accessed. For example, the phrenic nerve 28 may be accessed using an anterior approach. This is illustrated in FIG. 6, in which the cervical incision site 26 is located at the base of the neck 12 across the patient's sagittal plane or midline 93. This location allows for left, right, or bilateral access and stimulation if needed. In addition, the scar left by an incision at this location can be easily hidden within the folds of the patient's neck. Alternatively, the cervical incision site 26 may be located to the right or the left of the patient's midline 93 (see FIG. 7). FIG. 7 also shows a second incision site 94 for implanting the pulse generator (IPG) 38, which will be described later.

It is also contemplated that the phrenic nerve 28 may be accessed using a posterior approach in which the cervical incision site 26 is at a location that is posterior to the patient's subclavian triangle 88.

To locate the cervical incision site 26, the diagnostic ultrasound device 21 may be used to locate the phrenic nerve 28 and other nerves and major blood vessels near the subclavian triangle 88. The operator of the ultrasound device (US) 21 applies the ultrasound probe 22 to the neck 12, and the device 21 generates images shown on a display (not shown) of internal organs and tissue in the neck 12. The operator correlates the organs and tissue shown on the display to locations on the skin of the neck 12. The operator may mark the skin to indicate the path of the phrenic nerve 28. The operator may also mark the skin to indicate the position of other nerves and the major blood vessels. It is contemplated that the phrenic nerve 28 can be located by inserting an echogenic needle with a tip electrode (not shown) under ultrasound guidance to stimulate the phrenic nerve 28 with electric pulses.

The cervical incision site 26 may be selected to access a portion of the phrenic nerve 28 that is remote from the other nerves and major blood vessels. The location of the incision site 26 and the path from the incision to the phrenic nerve 28 may be selected to avoid damaging other nerves or major blood vessels and to minimize resection by following and bluntly separating muscles and the carotid sheath using natural boundaries formed by fascia and fat pads. Further, the path may be selected to avoid cutting muscle tissue and avoid using blunt dissection, using blunt tools and surgeons fingers, to the extent reasonably possible.

Once the cervical incision site is determined, an incision may be made (step 304). The incision may be in a range of two to five centimeters (cm), such as 3 to 4 cm. The specific length of the incision, such as within the range of 2 to 5 cm, may be determined by patient specific conditions such as obesity and neck circumference. For example, access to the phrenic nerve 28 for patients 10 with higher Body Mass Indices (BMIs) might be more challenging if trying to access the phrenic nerve 28 through a smaller incision.

For incisions 26 made across the patient's midline 93, the incision 26 may be perpendicular to the patient's midline plane 93. For incisions 26 made to the right or left of the patient's midline 93, the incision 26 may be 1-2 cm above and parallel to the patient's clavicle 39. As discussed above, FIGS. 6 and 7 show surgical incision sites 26. The incision 26 may be supraclavicular at the base of the subclavian triangle 88 of the neck 12. The target location may be in a region anatomically known as Level VB, which corresponds to the subclavian triangle 88.

It is contemplated that a low collar incision may be drawn on the anterior of the neck 12 and infiltrated with 1% lidocaine with 1/100,000 epinephrine. The skin of the neck 12 may be prepped and draped in the sterile manner. The skin may be incised with a #15 scalpel and the underlying platysma muscle was also incised with a scalpel.

For a posterior approach, the incision 26 may be made within a range of 0 to 1 cm posterior (behind) and parallel to the posterior border of the sternocleidomastoideole 32, and/or perpendicular direction to a line between the middle third of the clavicle 39 and the posterior border of the sternocleidomastoideole 32.

Once the incision 26 has been made, the incision 26 may be opened to expose the phrenic nerve 28 (step 306). For the anterior approach, the phrenic nerve 28 may be accessed by separating the planes between the sternocleidomastoideole 32 and the lateral side the internal jugular vein 36 toward the anterior scalene muscle 66.

Superficial bleedings due to the incision 26 may be controlled by electrocauterization. The platysma muscle, directly connected to the skin, is retracted to expose the underlying muscle and tissue. The incision 26 and retraction exposes an area between the clavicle 39, the omohyoid muscle 78, 80 and the posterior border of the sternocleidomastoideole 32. This area may be referred to a surgical opening (or incision well) 96 and is the specific anatomic space for dissection and accessing the phrenic nerve 28.

After the surgeon makes a blunt dissection of the subcutaneous adipose tissue (body fat under the skin), the sternocleidomastoideole 32 and the omohyoid muscle 78, 80 (which are separated by a tendon) are identified by the surgeon. To achieve overview and traction of the anatomic area (surgical opening 96) for improved dissection, the sternocleidomastoideole 32 is retracted in a medial direction and the omohyoid muscle 78, 80 is retracted in a lateral-superior direction using opposing forces applied by a surgical tool, such as a by tong (first and second retraction tools 30, 34), to generate mild traction of the tissue structures lying in between.

The retraction exposes the prevertebral fascia covering a portion of the anterior scalene muscle 66. The ventral surface of the anterior scalene muscle 66 that is dorsal to the lower third of the sternocleidomastoideole 32 is the layer upon which the electrode assembly 15 for nerve stimulation is to be residing after it is implanted and secured.

To surgically develop this layer, the surgical delivery tool 16 may include a distal end 98 optionally configured to provide blunt dissection (if needed). The distal end 98 of the surgical delivery tool 16 optionally includes an integrated neuromonitoring nerve detecting tip with feedback (e.g., acoustic, visual, or tactile) indicating close electric contact between the tip of the surgical delivery tool 16 and a neuronal structure.

As previously disclosed, the patient's neck 12 has many nerves, blood vessels and muscles in close proximity to each other that are at risk of being dissected or damaged during surgery. Thus, it is desired to minimize the size of the surgical opening 96 (or passage to the phrenic nerve 28 and anterior scalene muscle 66) to minimize damage to the surrounding nerves, blood vessels and muscles. However, the surgical opening 96 should be large enough so that the surgeon's view of the phrenic nerve 28 is not obstructed. Accordingly, it is contemplated that a width W of the surgical opening 96 (i.e., any span across the surgical opening 96) is within a range of 2-3 cm. It is further contemplated that the depth D of the surgical opening 96 is within a range of 5-15 cm.

Swabs or cold scissors may be applied to mobilize the fasciae surrounding the sternocleidomastoideole 32 and anterior scalene muscle 66. Retraction of the sternocleidomastoideocle 32 medially may be applied sequentially to progressively increase the size of the passage to the phrenic nerve 28 and anterior scalene muscle 66 as needed. Once the sternocleidomastoideole 32 is retracted, the phrenic nerve 28 with its perpendicular course should be clearly visualized over a length of 2-3 cm on the anterior surface of the anterior scalene muscle 66. Blunt dissection of tissue covering the phrenic nerve 28 and its fascia may be performed (if needed) with swabs to create a smooth surface on the prevertebral fascia and/or the anterior surface of the anterior scalene muscle 66 in regions on opposite sides of the phrenic nerve 28. The smooth surface may allow implantation and securement of at least one electrode assembly 15.

If an electrode is to be applied only over the phrenic nerve, the phrenic nerve 28 may remain in its sheath attached to the muscle and the fascia covering the nerve and need not be resected or directly, surgically manipulated or contacted. If a cuff electrode is to be applied to the phrenic nerve, a portion of the facia covering the nerve is surgically cut and a portion of the nerve is manipulated to allow a portion of the cuff electrode to be inserted through a tunnel formed between the nerve and a surface of the muscle below the nerve.

The phrenic nerve 28 may be accessed and exposed as described above at a location below the Erb's point, which is a junction of nerves originating from the cervical plexus. “Erb's point”, in the context of head and neck surgery, refers to a point on the posterior border of the sternocleidomastoideole 32 where the four superficial branches of the cervical plexus emerge from behind the muscle. Erb's point is a well-described anatomic landmark that can be readily located at the posterior border of the sternocleidomastoideole 32, located approximately at the border between the upper and middle thirds of the muscle. The incision may target the posterior border of the lower third of the sternocleidomastoideole 32.

Dissection may be carried anterior to the lower portion of the sternocleidomastoideole 32 along the medial border. The superficial layer of the deep cervical fascia may be grasped with hemostats and pulled medially while dissecting off the muscle. The internal jugular vein 36 may be identified, and dissection may be carried anterior to the internal jugular vein 36 in the supraclavicular area followed by the retraction of the internal jugular vein 36 to expose the phrenic nerve 28. This dissection may be carried inferior to the omohyoid muscle 78, 80. If supraclavicular fat is noted, this area may be defatted with electrocautery taking care not to injure the transverse cervical artery 46. Often times, the transverse cervical artery 46 will be identified crossing perpendicular and anterior to the phrenic nerve 28. The phrenic nerve 28 is identified passing inferiorly between the scalene muscles. This part of the phrenic nerve 28 is deeper than the deep layer of the deep cervical fascia.

Once the phrenic nerve 28 is exposed, the electrode assembly 15 may be inserted into the surgical opening 96 and positioned on the anterior scalene muscle 66 and across the phrenic nerve 28 (step 308). It is contemplated that surgical forceps or other common tools may be used to position the electrode assembly 15 on the anterior scalene muscle 66. However, such tools may be too bulky and cumbersome for the small space (surgical opening 96) in which the electrode assembly 15 is being positioned. The common tools may also obstruct the surgeon's view of the phrenic nerve 28 and the anterior scalene muscle 66. Accordingly, the surgical delivery tool 16 may be used to position the electrode assembly 15 into the surgical opening 96 instead of the common tools.

The surgical delivery tool 16 may be a device that is specially designed to facilitate the positioning of the electrode assembly 15 on the anterior scalene muscle 66 without obstructing the surgeon's view of the phrenic nerve 28 and the anterior scalene muscle 66. In particular, the surgical delivery tool 16 may have a smaller footprint than common tools. In addition, both the surgical delivery tool 16 and the electrode assembly 15 may be made of a transparent material (e.g., transparent polymer) in order to reduce the extent to which the surgical delivery tool 16 and the electrode assembly 15 obstruct the surgeon's view of the anterior scalene muscle 66 and the phrenic nerve 28 during the implanting of the electrode assembly 15.

Using the specially designed surgical delivery tool 16 to place and secure the electrode assembly 15 may conserve space within the surgical opening 96 by freeing the surgeon's hands to perform other functions. For example, the surgical delivery tool 16 may enable positioning and repositioning the electrode assembly 15 without having to insert the surgeon's fingers into the surgical opening 96 to manipulate the electrode assembly 15 and/or manipulate the muscles and nerves in the patient's neck 12.

The distal end 98 of the surgical delivery tool 16 may be configured to carry the electrode assembly 15 through the surgical opening 96 to the phrenic nerve 28 and the anterior surface of the anterior scalene muscle 66. During delivery of the electrode assembly 15 through the surgical opening 96, the surgical delivery tool 16 may be applied at the dorsal surface of the sternocleidomastoideole 32 and may be manipulated so that the electrode assembly 15 is positioned over the phrenic nerve 28 and onto the anterior surface of the anterior scalene muscle 66. To insert a cuff electrode, the surgical delivery tool may be configured to hold the electrode such that a distal end portion of the electrode extends from a distal end of the tool. The tool is manipulated such that the distal end portion of the electrode moves under the nerve. After moving under the nerve, the distal end portion of the electrode may be curved, foldable or biased to overlap nerve to encircle the nerve.

The surgical delivery tool 16 may be designed to enable attachment and detachment of the electrode assembly 15 inside the surgical opening 96. In addition, the surgical delivery tool 16 may be equipped with features to facilitate tacking the electrode assembly 15 to the anterior scalene muscle 66. After the electrode assembly 15 has been tacked to the anterior scalene muscle 66 by the surgical delivery tool 16 and while the electrode assembly 15 is still attached to the surgical delivery tool 16, the external current generator 18 may generate a test stimulation current that is discharged to the phrenic nerve 28 through the electrode assembly 15 (step 310).

Referring back to FIG. 1, the external current generator 18 may be positioned away from the patient and may be positioned at either a sterile location inside the surgical field or a non-sterile location outside of the surgical field. Thus, the lead extension 17 may be used to bridge the distance between the electrode assembly 15 and the external current generator 18. In particular, one end of the lead extension 17 may be connected to the external current generator 18, while the other end of the lead extension 17 may be connected to the wire or lead 41 of the electrode assembly 15. It is contemplated that the lead extension 17 may comprise a plurality of wired subsections connected in series. It is further contemplated that all or part of the lead extension 17 may be integrally formed with the wire or lead 41 of the electrode assembly 15.

The test stimulation current can be actuated manually or automatically. For example, once the external current generator 18 is turned on, the test stimulation current may be automatically actuated by the external current generator 18 based on timing or other parameter. Alternatively, the test stimulation current may be actuated by the surgeon engaging a user interface on the surgical delivery tool 16 or on the external current generator 18. The user interface may be a button, a dial, a trigger, a lever, or any other device that may convert a physical input from the surgeon to an analogue or digital input to the external current generator 18. It is contemplated that the generation of the test stimulation current may be voice activated by way of, for example, a microphone.

The magnitude of the test stimulation current may be manually or automatically selected. In one example, bipolar constant current pulses of 100-500 microseconds long can be delivered to the phrenic nerve 28 at a frequency of 20-50 Hz. After the current is maintained at a constant frequency the voltage may be selected depending on the tissue impedance, which can be in the range of 100 to 1,000 ohm. Once the pulse length, frequency and voltage are selected, the test stimulation current may be ramped up until a first diaphragmic contraction is detected (motor neuron capture) (e.g., within a range of 1.0 to 5.0 mA). The detection may be made by (for example) visually looking at the patient for signs of diaphragmatic contraction, looking at signals on a monitor generated by various sensors, or listening to audible signals generated by the various sensors.

Detection can be made by an impedance tension belt 23 or an accelerometer. It can be anticipated that pulse trains of stimulation energy can be discharged at a constant pulse rate that is easy to detect, such as, for example, 10-30 pulses per minute. Energy is then increased further in steps until the full engagement of all nerve fibers in the phrenic nerve 28 and muscle fibers in the anterior scalene muscle 66, after which, no more muscle contraction results from the further increase of energy (tetanized muscle). The diaphragmatic contraction may be measured by way of an esophageal balloon pressure, respiratory belts, transthoracic impedance, magnetometry, accelerometry and inspiratory pressure measurement.

The surgeon may analyze the resulting data to determine whether the electrode assembly 15 is correctly positioned (step 312). In particular, the electrode assembly 15 may be considered to be correctly positioned when the electrode assembly 15 is in the position with the lowest capture threshold, i.e., the minimal current needed to cause the diaphragmic muscle contraction. If the electrode assembly 15 is not in a position with the lowest capture threshold, the surgeon may reposition the surgical delivery tool 16 with the electrode assembly 15 to a new position, resecure the electrode assembly 15 to the anterior scalene muscle 66 and apply another test stimulation current to the phrenic nerve 28 (repeat steps 308 and 310).

It is contemplated that the lowest capture threshold may be determined after the surgeon has repositioned the surgical delivery tool 16 and the electrode assembly 15 one or more times. For example, after the surgical delivery tool 16 and the electrode assembly 15 has been repositioned multiple times. For example, it may be determined that the minimal nerve capture current is 1.0 mA. The minimal nerve capture current may be determined to be the lowest capture current after performing a predetermined test stimulation currents at a predetermined number of different locations.

Once the minimal nerve capture current is determined, the surgical delivery tool 16 and the electrode assembly 15 may be repositioned to the location at which the minimal nerve capture current was experienced. After the surgical delivery tool 16 and the electrode assembly 15 have been positioned at the elected location, a higher current may be applied to the phrenic nerve 28 to cause total engagement and tetanus of the anterior scalene muscle 66 (step 314). For example, the anterior scalene muscle 66 may be fully tetanized at 5 mA. This value may be set to be the maximum limit for the stimulation current applied to the phrenic nerve 28 to ensure that other muscles in the neck or shoulders are not engages at the elected position.

It is contemplated that the parameter values discussed above can be used as dependent variables in the creation of a therapy titration curve which can be used to program the settings of the IPG 38.

After the position of the electrode assembly 15 has been confirmed to be correct, the electrode assembly 15 may be safely glued, tacked, or stapled to the anterior scalene muscle 66 (step 316).

This may be performed by a fixation tool 20. For example, the fixation tool 20 may be inserted into the surgical opening 96 while the electrode assembly 15 is being held in place by the surgical delivery tool 16. The fixation tool 20 may then engage the surgical delivery tool 16 and the electrode assembly 15 to glue, tack, or staple, the electrode assembly 15 to the anterior scalene muscle 66.

Once the electrode assembly 15 is secured to the anterior scalene muscle 66, the fixation tool 20 and the surgical delivery tool 16 are removed from the surgical opening 96. In addition, the wire or lead 41 is disconnected from the external current generator 18 (step 318). The disconnection may be achieved by disconnecting the wire or lead 41 from all or part of the lead extension 17.

A subcutaneous pocket for the IPG 38 may be formed in the pectoral region at the second incision site 94 (step 320). The incision may be 5-6 cm and may be located 2-3 cm below the mid-portion of the right clavicle 39. In addition, the subcutaneous pocket may be of sufficient size to contain the IPG 38 and any excess wire or lead 41 for strain relief. It is contemplated that the subcutaneous pocket may be located at the patient's armpit or other location instead of the pectoral region. Either way, the subcutaneous pocket should be sized to allow for minimal or no movement of the IPG 38 and aesthetic considerations.

After the subcutaneous pocket for the IPG 38 has been created and the IPG 38 has been implanted, a subcutaneous passage from the electrode assembly 15 to the subcutaneous pocket may be formed (step 322). A tunneling tool (not shown) may be employed to create the subcutaneous passage.

Once the subcutaneous passage is created, the wire or lead 41 may be routed through the subcutaneous passage and connected to the IPG 38 (step 324). In particular, the lead or wire 41 may be carried across the trachea 84 over the suprasternal notch then obliquely through a tunnel to the lateral aspect of the chest. In addition, one or more strain relief devices 42 may anchor a portion of the lead or wire 41 to a muscle or connective tissue to prevent the lead 41 from being pulled out of the IPG 38 and/or prevent the lead 41 from pulling the electrode assembly 15 out of position. It is contemplated that the strain relief device 42 may be within the incision at the cervical incision site 26 instead of in the second incision site 94 (FIG. 2). Also, there may be strain relief devices 42 at both the cervical incision site 26 and the second incision site 94.

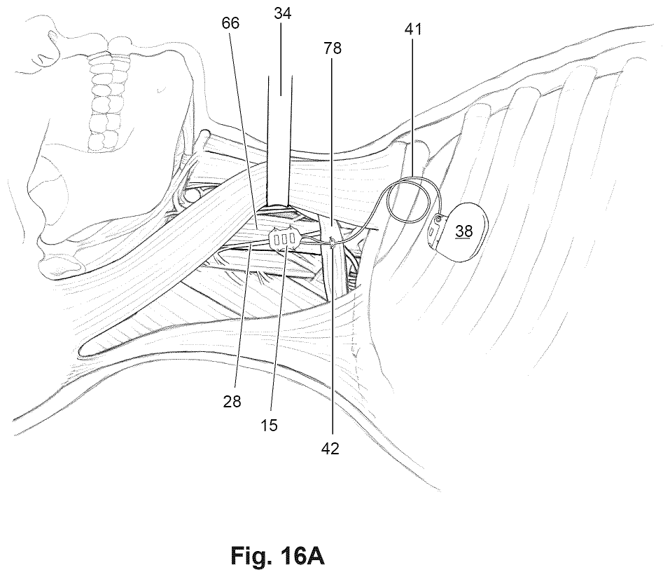

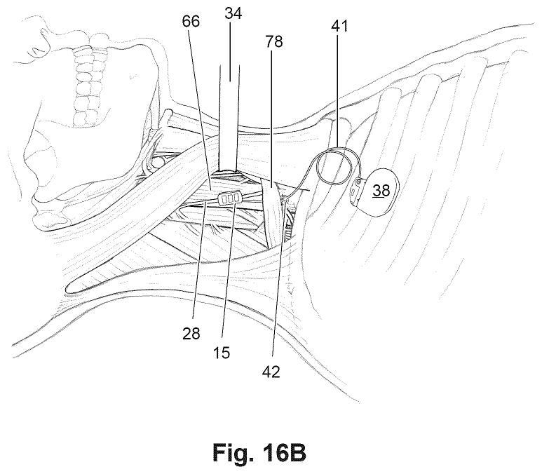

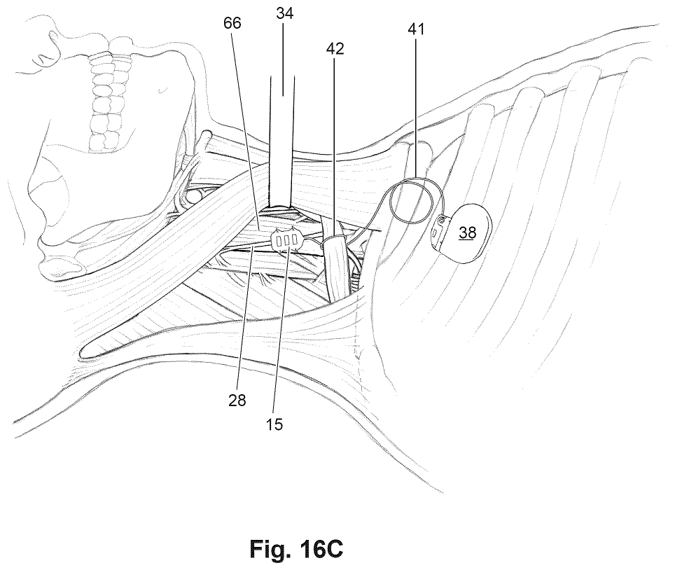

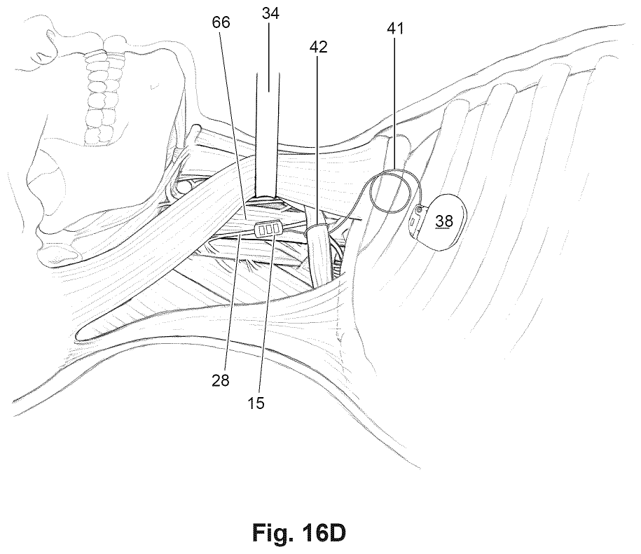

As shown in FIGS. 16A and 16B, a strain relief device 42 may be anchored to the omohyoid muscle 78 by way of, for example, suturing. The omohyoid muscle 78 may serve as a desirable anchoring structure because the omohyoid muscle 78 is less mobile than other anatomical structures inside the patient's neck. It is contemplated that other anatomical structures inside the neck that are relatively stationary may also serve as anchor points for the strain relief device 42. For example, it may be desirable to suture the strain relief device 42 to connective tissue such as tendons. In addition, the strain relief device 42 may be sutured to the omohyoid muscle 78 or other anatomical structure regardless of the type of electrode being used (e.g., a paddle electrode (FIG. 16A) or a cuff electrode (FIG. 16B)).

As shown in FIGS. 16C and 16D, the strain relief device 42 may be wrapped around the omohyoid muscle 78 instead of being sutured or fixed to the omohyoid muscle 78. When being wrapped around an anchoring structure, the strain relief device 42 may form one or more loops. Wrapping the strain relief device 42 around the omohyoid muscle 78 allows the strain relief device 42 to allow some movement of the omohyoid muscle 78 and still prevent or minimize a pulling of the electrode assembly 15 by the lead 41. In particular, because the strain relief device 42 is wrapped around the omohyoid muscle 78 instead of being fixed to the omohyoid muscle 78, the strain relief device 42 may slide along omohyoid muscle 78 while the neck and the omohyoid muscle 78 move. This may allow the distance between the strain relief device 42 and the electrode assembly 15 to remain substantially constant during movement of the neck. It may also allow the length of the lead 41 between the strain relief device 42 and the electrode assembly 15 to increase or decrease (as the lead 41 winds or unwinds around the anchoring structure as the anchoring structure moves) to accommodate the movement of the anchoring structure.

The strain relief device 42 may be wrapped around other anatomical structures in the neck. For example, it may be desirable to wrap the strain relief device 42 around connective tissue such as tendons. In addition, the strain relief device 42 may be wrapped around the omohyoid muscle 78 or other anatomical structure regardless of the type of electrode being used (e.g., a paddle electrode (FIG. 16C) or a cuff electrode (FIG. 16D)). It is also contemplated that the strain relief device 42 may be both sutured to the anchoring structure and wrapped around the anchoring structure.

Once the lead 41 is connected to the IPG 38, the IPG 38 may provide electrical signals that are transmitted to the electrode assembly 15 and are applied to stimulate the phrenic nerve 28. The IPG 38 may also house a sealed battery and may include an antenna to receive power and communicate to devices external to the body of the patient 10. The devices may include a recharger that recharges the battery and a processor configured to receive data from the pulse generator and to transmit, for example, updates to algorithms for stimulating the phrenic nerve.

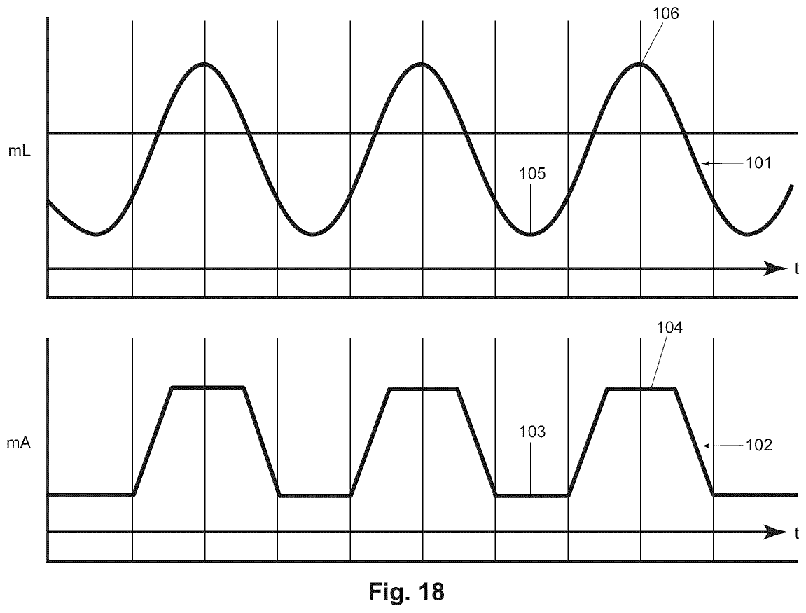

The IPG 38 may be in communication, e.g., wired or wirelessly, with other devices, such as devices sensing respiration of the patient and body position and movements of the patient during sleep and while awake. These devices may be implanted in the patient body, wearable devices or even external to the patient altogether such as microwave radar devices used to detect breathing and motion. Data from these other devices may be used by the embedded intelligence in the pulse generator 38 or an external processor device to determine when and whether and how much to simulate the phrenic nerve 28 to induce the diaphragm to contract, stay contracted or contract further than would occur without the stimulation. These programs are generally called waveform generation. An example of a therapy waveform is given in FIG. 18.

Once the wire or lead 41 is connected to the IPG 38, the connection may be tested by transmitting electrical stimulation signals to the electrode assembly and observing the waveforms on an external monitor (step 326). The external monitor may be a programmable device integrated with a tablet or even a remote device. When the stimulation and sensing functions are confirmed, both incisions 26, 94 may be closed (step 328). The cervical incision 26 may be closed in multiple layers with absorbable sutures in a subcuticular manner and the surface of the skin is closed with glue.

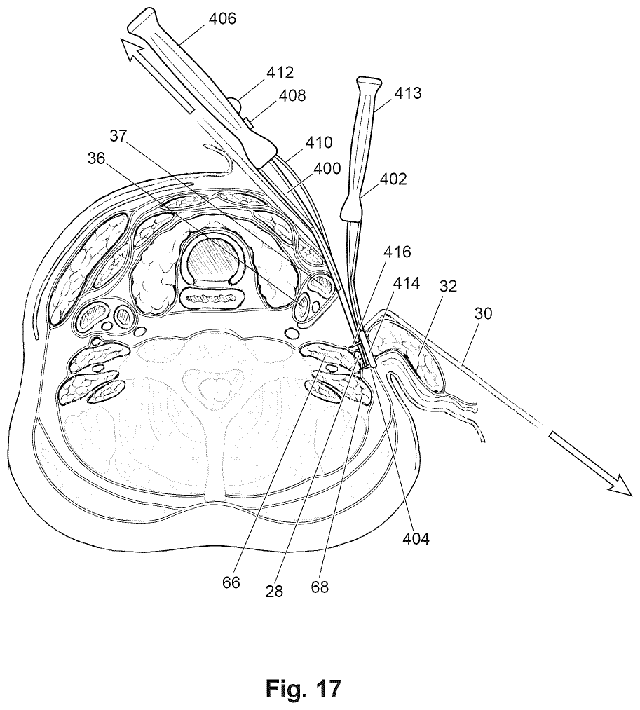

FIG. 17 illustrates another embodiment of a delivery tool 400 and fixation tool 402 used to implant an electrode 15 over a phrenic nerve 28. The delivery tool 400 and the fixation tool 402 may be similar to the delivery tool 16 and the fixation tool 20 except that the external current generator is located inside the delivery tool 400. Accordingly, the delivery tool 16 and the delivery tool 400 may share some features. In addition, the fixation tool 20 and the fixation tool 402 may share some features.

Locating the external current generator in the handle of the delivery tool 400 instead of outside the sterile surgical environment may simplify the system and may reduce the possibility of a breach of the sterile environment. In particular, the lead extension 17 may be connected to an electrical connector in the handle 406 of the delivery tool 400 instead of being routed to a location outside of the sterile environment. In other words, locating the external current generator in the delivery tool 400 does not require the sterile barrier to be pierced by the lead extension 17. It is also contemplated that locating the external current generator in the delivery tool 400 may allow for the lead 41 to be directly connected to the external current generator, thereby eliminating the need for a lead extension 17. It is further contemplated that the delivery tool 400 with the self-contained electrical equipment may be a one-use (or disposable) device.

Also, the delivery tool 400 may be provided in a kit wherein the delivery tool 400 is enclosed in a sterile packaging. The sterile packaging may also include the fixation tool 402. The kit may be a one-use (disposable) kit. Alternatively, the tools in the kit may be reusable in another sterile kit after undergoing a cleaning and sterilizing process.

A distal end 404 of the delivery tool 400 may include a front surface configured to temporarily support an electrode. A physician may manually maneuver the handle 406 of the delivery tool 400 to position the distal end 404 with the electrode 15 over the phrenic nerve 28 and the anterior surface of the anterior scalene muscle 66.

To confirm that the electrode 15 is positioned over the phrenic nerve 28, the physician may push a button 408 (or actuate another type of user input such as a switch, knob, or voice activation device) on the handle that actuates a battery powered electronic circuit within the handle to apply electrical power to the electrode via a conductive lead 410. The lead 410 may be releasably attached to the delivery tool 400 and may extend from the handle 406 along the arm of the tool 400 to the distal end 404 and the electrode 15.

An indicator 412 in the form of a light source, audible alert or other indicator mechanism (either part of the delivery tool 400 or separate from the delivery tool 400), may signal to the physician that electrical power is applied to the electrode 15. For example, the indicator 412 may emit a sound that changes frequency, may emit light that changes color, and/or may generate a repeating signal (audible, visual, or tactile) that changes its periodicity depending on the positioning of the electrode 15 relative to the phrenic nerve 28.

The physician may also monitor the patient 10 to determine if the phrenic nerve 28 is stimulated by electrical current flowing from the electrode 15 into the phrenic nerve 28. The monitoring of the patient 10 may include observing the patient's chest cavity movement (or signals from sensors indicative of movement of the patient's chest cavity) in response to movement of the patient's diaphragm or a change in the patient's breathing pattern. The physician may reposition the electrode 15 over the anterior scalene muscle 66 until the electrode 15 is stimulating the phrenic nerve 28 (similar to the procedure disclosed with the delivery tool 16).

When the electrode 15 is properly positioned over the phrenic nerve 28, the physician may maneuver a handle 413 of the fixation tool 402 to align a distal end 414 of the fixation tool 402 with the distal end 404 of the delivery tool 400 and the electrode 15 held on the distal end 404. The distal end 414 of the fixation tool 402 may releasably hold fasteners 416, e.g., screws, staples, sutures or nails. These fasteners 416 are aligned with holes in the electrode 15 and in the distal end 404 of the delivery tool 400 by maneuvering the handle 413 of the fixation tool 402.

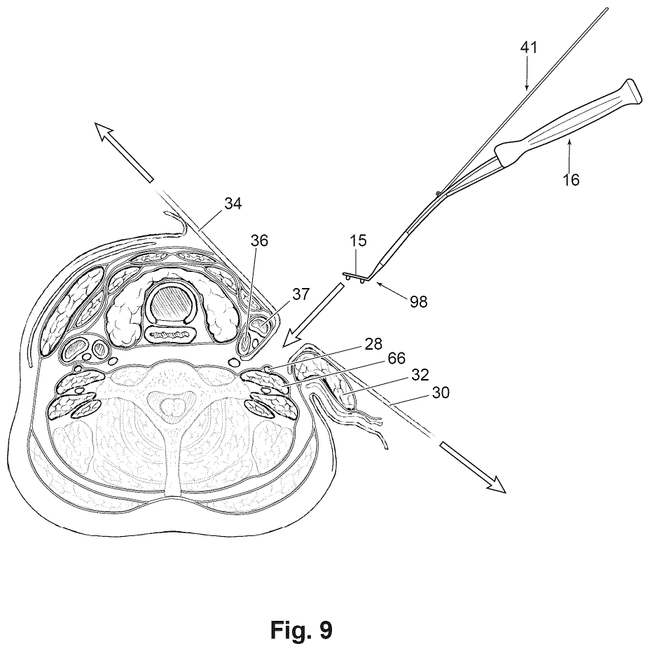

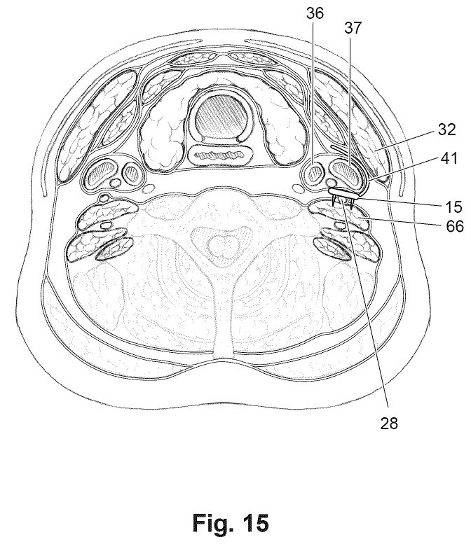

By pressing the distal end 414 of the fixation tool 402 against the distal end 404 of the delivery tool 400, the fasteners 416 may be inserted through the holes in the distal end 404 of the delivery tool 400 and may be embedded in the anterior scalene muscle 66. The fasteners 416 may secure the electrode 15 to the anterior scalene muscle 66 and may position the electrode 15 over the phrenic nerve 28. The fasteners 416 may also assist in releasing the electrode 15 from the delivery tool 400 as that tool 400 and the fixation tool 402 are removed from the patient 10. The fasteners 416 may be formed of a biodegradable material that dissolves within a few months and after fibrous tissue forms over the electrode 15 and secures the electrode 15 over the phrenic nerve 28. It should be understood that similar fasteners may be used with the system illustrated in FIGS. 9-16D.

Once the electrode assembly 15 is secured to the anterior scalene muscle 66, the fixation tool 402 and the surgical delivery tool 400 are removed from the surgical opening 96. In addition, the wire or lead 41 is disconnected from the external current generator 18 (i.e., disconnected from the delivery tool 400 and/or the handle 406). Alternatively, the disconnection may be achieved by disconnecting the wire or lead 41 from all or part of the lead extension 17.

Once the lead 41 is disconnected from the delivery tool 400, steps 320 through 330 of the method illustrated in FIGS. 5-15 may be performed. In addition, the strain relief devices 42 may be positioned as disclosed above.

FIG. 18 illustrates a bilevel stimulation waveform applied by the IPG 38 to the phrenic nerve 28 in a patient 10 with sleep apnea. The upper waveform 101 illustrates an inhalation and exhalation of a patient 10 when the patient's airway is unobstructed. The bottom waveform 102 illustrates stimulation pulses applied to the phrenic nerve 28 to achieve entrainment of breathing and increase lung volume. It is believed that increased lung volume during exhalation generates caudal traction on the airway and helps keep the airway open during expiration. In this example, the patient's breathing rate is entrained, i.e., synchronized to a driving stimulation rhythm that can be ten breaths per minute but spontaneously and naturally generated by the patient's central nervous system and peripheral reflexes.

Entrainment of natural breathing rhythms by stimulation of phrenic nerve to treat Central Sleep Apnea (CSA) is described in U.S. Pat. No. 9,370,657 to Tehrani, which describes therapy that combines manipulating tidal volume, biasing the lung volume and breathing entrainment.

The entrainment discussed above may be applied to the treatment of OSA by application of at least two levels of stimulation: inspiratory level 103 and expiratory level 104 and maintaining natural respiration whilst regularizing the patient's breathing to the rate set by the timing of the stimulation while maintaining two corresponding levels of lung distension: inspiratory volume and expiratory “bias” volume. The duty cycle or inspiratory to expiratory (I:E ratio) time ratio can be set by a physician or adjusted automatically if excessive air trapping is detected. In order to be practical, such therapy may need be adaptive where both inspiratory and expiratory period stimulation levels can be automatically adjusted based on patient's respiration and body position.

The bilevel entrainment stimulation illustrated in FIG. 18 can treat a variety of conditions that often accompany OSA such as obesity induced hypoventilation, central sleep apnea and mixed sleep apnea (an apnea type where airway instability is accompanied by the instability of respiratory drive). The bilevel entrainment stimulation can be also instrumental in titration and auto-titration of phrenic stimulation therapy.

The upper waveform 101 illustrates respiratory airflow over time with the inspiration phase 105 being followed by the expiration phase 106. The lower waveform 102 illustrates the stimulation of the phrenic nerve 28. Stimulation phases are set to a programmed frequency, which can be between 6 and 20 bpm (0.1 and 0.33 Hz) that is the physiologic range where the patient's natural breathing can be entrained by stimulation, which means that patient synchronizes the onset of natural inspiration, including airway muscles and respiratory pump to the inspiratory level 103 of stimulation energy. The inspiratory level 103 is lower than the expiratory level 104 and is selected to maintain the bias of the lung and a predetermined end expiratory lung volume to prevent airway collapse according to other aspects of the invention.

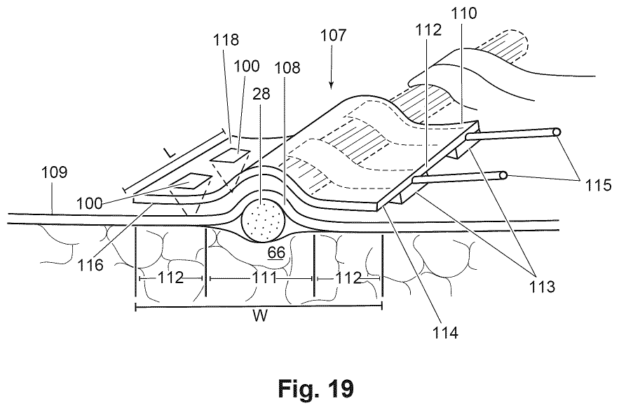

FIG. 19 is a perspective view of an exemplary paddle or flexible plate electrode assembly 107 placed over the phrenic nerve 28 and the fascia 108 covering the phrenic nerve 28 and the anterior surface of the anterior scalene muscle 66. Many alternative shapes and designs can be envisioned but commonly, the electrode assembly 107 may include a flexible dielectric panel (substrate) 110 that forms a structural support for the electrode assembly 107 and may be oriented such that the phrenic nerve 28 underlies approximately a center portion 111 of the electrode assembly 107 to allow for some motion during placement. The center portion 111 may extend the length (L) of the electrode assembly 107. Side portions 112 of the electrode assembly 107 may also extend the length of the electrode assembly 107. The center portion 111 is sandwiched between the side portions 112.

Electrodes 113, such as conductive strips, conductive pads or cuffs, may be attached, electrochemically deposited upon or at least partially embedded in a back side 114 of the flexible panel 110, wherein the back side faces the phrenic nerve 28 and anterior surface of the anterior scalene muscle 66. The flexible panel 110 may be formed of a biocompatible dielectric material, such as PI (Polyimide). A dielectric material is an insulator that blocks electrical current. Thus, current flows from the electrodes 113 away from the panel 110 and towards the phrenic nerve 28. By directing current towards the phrenic nerve 28 and blocking current flowing away from the phrenic nerve 28, the panel 110 concentrates the current flowing to the phrenic nerve 28 and thereby reduces the electrical energy needed to be produced by the pulse generator 38 to stimulate the phrenic nerve 28.

The back side 114 of the panel 110 which faces the phrenic nerve 28 may be directly affixed to prevertebral fascia 109 and/or the anterior surface of the anterior scalene muscle 66 by way of the fasteners 100. The fasteners 100 may be similar to the fasteners 416 that were previously described.

The electrodes 113 may be noble metal conductors embedded in the flexible panel to provide a fully biocompatible platform for the long term implanted stimulation of the nerve that will not significantly degrade over years of use. Electrode surface coatings can be composed of noble conductive metals such as Pt, PtIr, IrO2 or Ti. Surface coatings may be electroplated or sputtered. Conductors can be recessed or fully embedded in biocompatible dielectrics. The electrode strips 113 can be recessed or raised to the polymer surface. Minimum substrate thickness that can be manufactured using modern methods is approximately 50 μm. Currently used flexible substrates for implantable electrode systems include silicone, polyimide (PI), polydimethylsiloxane (PDMS), parylene, among others. For example, PI film is a high-temperature resistant polymer film that served as a medical implantable material has a long history but it is anticipated that material science is rapidly advancing and other superior substrate materials may be available as well as layers of materials combined and fused together can produce advanced physical and biophysical properties.

The electrodes 113 may be adapted to minimize losing nerve capture if the electrode 113 is slightly moved in relation to the phrenic nerve 28 such as if it turns or slides somewhat after the release or deployment by the surgeon. An approach to minimizing losing nerve capture is to form the electrodes 113 as conductive strips having a length extending at least a majority of a width W of the flexible panel 110, wherein the width W includes the sides 112 and central portion 111 of the flexible panel 110, as shown in FIG. 19. Alternatively, the electrodes 113 may extend a majority of the length (L) of the panel 110. The dimensions of the electrode assembly 107 and its components are constrained by the dimensions of the surgical opening 96, which are disclosed above.

Conductive leads 115 connect the electrodes 113 to the wire(s) inside the lead 41 that extends to the pulse generator 38. The electrodes 113 are electrically isolated from each other by the dielectric properties of the panel 110. The dielectric properties of the panel 110 also direct the electric field generated by the electrodes 113 towards the phrenic nerve 28. The dielectric properties of the panel 110 prevent current flow through the panel 110 and thus prevents current flow away from the phrenic nerve 28. Because the panel 110 directs current towards the phrenic nerve 28 and prevents current flow away from the nerve, the current is concentrated by the panel 110 to flow towards the phrenic nerve 28. This concentration of current reduces the electrical energy that is needed to apply current to stimulate the phrenic nerve 28.

The electrodes 113 may be separated by a gap in a range of 1mm to 10 mm that extends the length of the strips forming the electrodes 113.

Current moves between the electrodes 113 by flowing through the phrenic nerve 28, fascia, and muscle underling the electrode 113. It is anticipated that a fibrotic capsule will form around the foreign body in the weeks following the implant and will become part of the current flow path. The current flow through the phrenic nerve 28 is sufficient to cause action potential and stimulate the nerve, which thereby causes the individually innervated muscle fibers in the diaphragm to contract or contract to a greater extent than would naturally occur during the respiratory cycle. It is generally accepted that the amount of electric energy (dependent on electric current, pulse duration and frequency) delivered to the excitable nerve tissues will activate more fibers until the muscle is “fused” and completely contracted or tetanized and can contract no more.

The electrode assembly 107 may be positioned directly on the anterior surface of the anterior scalene muscle 66 with the phrenic nerve 28 below the central portion 111 of the electrode assembly. The electrode assembly 107 may include a fibrosing surface(s) 116 on the back side of the panel 110 of the electrode assembly 107, such on both sides 112 of the panel 110. The fibrosing surface(s) attach the electrode assembly 107 to the anterior scalene muscle 66. The fibrosing surface(s) 116 promotes fibrotic tissue formation at the interface between the electrode assembly 107 and the anterior surface of the anterior scalene muscle 66 that naturally occurs as a foreign body response to anchor the electrode assembly 107 to the anterior scalene muscle 66. The fibrosing surface 116 on the electrode assembly 107 may include layer of porous material that encourages ingrowth of tissue, a medical adhesive that adheres to muscle, and micro-needles, micro-gripping hydrophobic patterns or gecko feet pattern, that extend from a planar surface of the electrode assembly 107 and are embedded into the surface of the anterior scalene muscle 66. For example, micro porous Gore-Tex expanded PTFE is a biocompatible structure into which cells can penetrate. This material is incorporated into the surrounding tissue, not encapsulated by it.

Implantation of the electrode assembly 107 may or may not require removal the fascia sheath 108 covering the phrenic nerve 28, separating the phrenic nerve 28 from the surface of the anterior scalene muscle 66 or tunneling under the phrenic nerve 28 and between the phrenic nerve 28 and the anterior scalene muscle 66. However, a cuff electrode will require tunneling under the phrenic nerve.

The fibrosing surface 116 of the electrode assembly may be confined to the back side 114 of the panel 110 of the electrode assembly 107. The back side 114 faces and attaches to the anterior scalene muscle 66. The front surface 118 of the panel 110 may be anti-fibrotic, such as having a low friction, non-irritating, non-adhesive smooth coating or layer. A low friction, non-adhesive smooth coating or layer may be silicone, hydrogel, PTFE (polytetrafluoroethylene) or a hydrophilic coating.

The front surface 118 faces towards and is adjacent the sternocleidomastoideole 32 and possibly other anatomic structures or tissue layers. There may be relative movement between the anterior scalene muscle 66 and the sternocleidomastoideole 32 and other anatomic structures or tissue layers adjacent the front surface 118 of the electrode assembly 107. The relative movement should not move the electrode assembly 107 from its position on the anterior scalene muscle 66 and over the phrenic nerve 28. A low friction non-adhesive coating or layer on the front surface 118 of the electrode assembly 107 allows the electrode assembly 107 to remain stably positioned on the anterior scalene muscle 66 and over the phrenic nerve 28, while the front surface 118 of the electrode assembly 107 slides with respect to the sternocleidomastoid muscle 32 and other anatomic structures or tissue layers adjacent the front surface 118 of the electrode assembly 107.

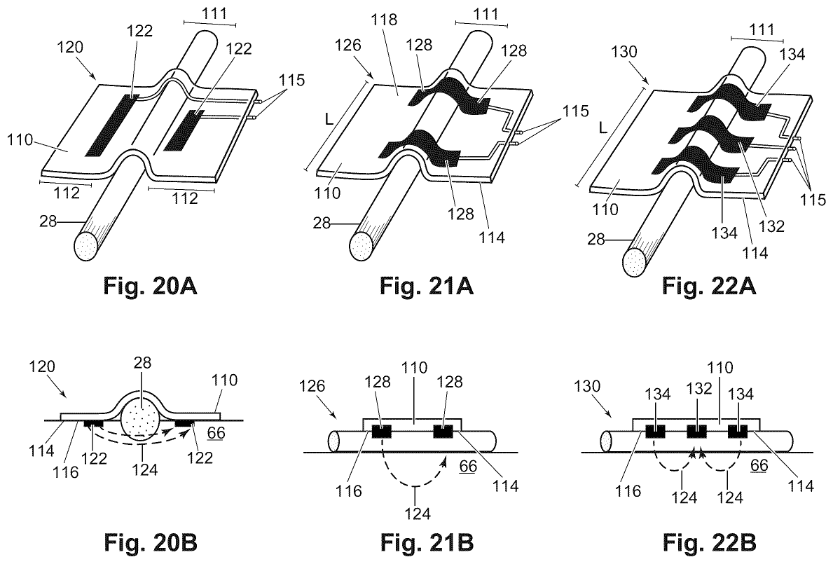

FIGS. 20A and 20B show an embodiment of a bi-pole electrode assembly 120 having electrodes 122, e.g., conductive strips, parallel to each other and arranged on the sides 112 of the electrode assembly 120 and on opposite sides of the center portion 111. FIG. 20A is a perspective view of the bipolar electrode assembly system 120 overlying the phrenic nerve 28 where one electrode 122 forms a cathode and the other electrode 122 forms an anode. The electrodes 122 may be metal strips parallel to the phrenic nerve 28 and positioned on two sides of the phrenic nerve 28. FIG. 20B is an end view of the bipolar electrode assembly 120 and a cross-sectional view of the phrenic nerve 28. The electrodes 122 may be oriented and extend in a lengthwise direction of the electrode assembly. The electrodes 122 may be embedded or mounted to the back side 114 of the flexible panel 110 of the electrode assembly 120. The electrodes 122 are generally parallel to and on opposite sides of the phrenic nerve 28. The electrodes 122 do not need to overlie the phrenic nerve 28. Current 124 from the conductive leads 115 flows from one of the electrodes 122, through the phrenic nerve 28 and adjacent tissue and to the other electrode 122. This current flow stimulates the phrenic nerve 28. The dielectric properties of the flexible panel 110 block current flow away from the phrenic nerve 28.

FIGS. 21A and 21B show an embodiment of a bi-pole electrode assembly 126 similar to the electrode assembly 107 shown in FIG. 18. FIG. 21A is a perspective view of the electrode assembly 126 overlying the phrenic nerve and FIG. 21B is a side view of the electrode assembly 126 and electrodes 128. The electrodes 128 are parallel to each other and arranged to span across the center portion 111 of the electrode assembly 126 in a direction generally perpendicular to the length (L) of the electrode assembly 126. The electrodes 128 are embedded or mounted to the back side 114 of the flexible panel 110 of the electrode assembly 126. The electrodes 128 extend over the phrenic nerve 28. Current from the conductive leads 115 flows from one of the electrodes 128, through the phrenic nerve 28 and adjacent tissue and to the other electrode 128. This current flow stimulates the phrenic nerve 28. The dielectric properties of the flexible panel 110 block current flow away from the phrenic nerve 28.

FIGS. 22A and 22B show an embodiment of a tri-pole electrode assembly 130. FIG. 22A is a perspective view of the electrode assembly 130 overlying the phrenic nerve 28, and FIG. 22B is a side view of the electrode assembly 130 and electrodes 132, 134, e.g., conductive strips. The electrodes 132, 134 may be parallel to each other and arranged to span across the center portion 111 of the electrode assembly in a direction generally perpendicular to the length (L) of the electrode assembly. The electrodes 132, 134 are embedded or mounted to the back side 114 of the flexible panel 110 of the electrode assembly. The electrodes 132, 134 extend over the phrenic nerve 28. The electrodes 132, 134 may overlie the phrenic nerve 28. The outer electrodes 134 may be connected, via leads 115, to a voltage source in the pulse generator 38 and the center electrode 132, e.g., conductive strip, may be connect to neutral, e.g., ground of the pulse generator. Current 124 flows through the phrenic nerve 28 by flowing between the outer electrodes 134 and the center electrode 132. This current 124 stimulates the phrenic nerve 28. The dielectric properties of the flexible panel 110 block current flow away from the phrenic nerve 28.

The logic inside the IPG 38 can allow selection of a stimulation pattern where the central electrode 132 is a stimulating cathode or any other combination including monopolar and bipolar stimulation patterns. Each configuration is composed of a unique active site (cathode) on the central electrode and several return path (anode) for which the current ratio is imposed which can be the IPG metal case itself which is commonly called monopolar configuration since the relatively large IPG case acts as a dispersing electrode.

The pulse generator 38 may also be configured to deliver two anodic currents for tri-polar nerve stimulation where the center electrode 132 acts as a cathode and the outer electrodes 134 act as anodes.

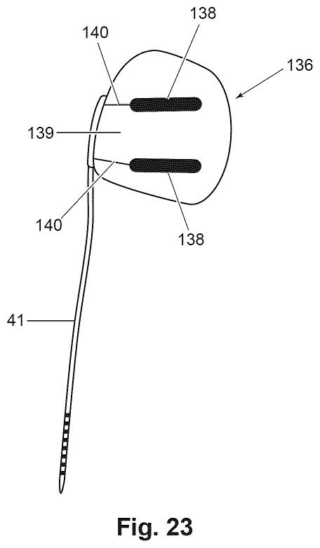

FIG. 23 illustrates another paddle-type electrode assembly 136. Similar to the electrode assembly 107, the electrode assembly 136 may include a pair of electrodes 138 positioned on a flexible, transparent dielectric panel (substrate) 139.

The electrodes 138 may each be a unilateral bipolar electrode in the form of a conductive strip. Each strip may extend in parallel to each other so that each electrode 138 overlaps the phrenic nerve 28 when the phrenic nerve 28 underlies a central part of the flexible panel 139. The electrodes 138 may be attached, electrochemically deposited upon, or at least partially embedded in a side of the flexible panel 139 that contacts the anterior scalene muscle 66.

It is contemplated that the electrodes 138 and the flexible panel 139 may be formed in a manner similar to that which is disclosed for the configuration illustrated in FIG. 19. The electrodes 138 and the flexible panel 139 may also be formed from the same material disclosed above for the configuration illustrated in FIG. 19.

Conductive leads 140 connect the electrodes 138 to the wire(s) inside the lead 41 that extends to the pulse generator 38. It is contemplated that the conductive leads 140 may be positioned on or embedded within the panel 139 and may extend to an edge of the panel 139. The lead 41 may terminate at the edge of the panel 139.

The electrode assembly 136 may be anchored to the anterior scalene muscle 66 by way of sutures, micro needles, micro-hooks, micro-teeth, a micro-patterned dry adhesive and/or adhesive, or any other biocompatible means of securing the electrode assembly 136 to the anterior scalene muscle 66. These attachment aids can be biodegradable and eventually bioabsorbed into the body since the assembly in time will be held in place by fibrosis of tissues.

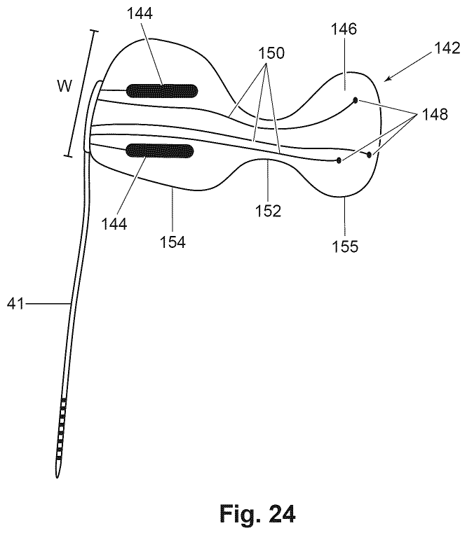

FIG. 24 illustrates another paddle-type electrode assembly 142 that is configured to stimulate the phrenic nerves 28 and sense a volume of air inside the patient's airways and lungs. Similar to the electrode assembly 136, the electrode assembly 142 comprises a pair of electrodes 144 positioned on (or embedded within) a flexible, transparent dielectric panel (substrate) 146. The electrodes 144 may each be a unilateral bipolar electrode in the form of a conductive strip. The electrode assembly 142 may also include one or more impedance sensors 148 (e.g., tripolar tracheal flow impedance sensing leads) positioned on (or embedded within) the panel 146. The impedance sensors 148 may be positioned to sense a volume of air inside the patient's airways and lungs.

Conductive leads 150 may connect the electrodes 144 and the impedance sensors 148 to the wire(s) inside the lead 41 that extends to the pulse generator 38. It is contemplated that the conductive leads 150 may be positioned on or embedded within the panel 146 and may extend to an edge of the panel 146.

The panel 146 may include a central narrowed portion 152 between a first lateral widened portion 154 and a second lateral widened portion 155. The width W of the panel 146 at the central narrowed portion 152 may be less than the width W of the panel 146 at the first and second lateral widened portions 154 and 155. This shape may allow for a more robust securement of the electrode assembly 142, while minimizing the footprint of the electrode assembly 142 to accommodate the small crowded space in the patient's neck 12. However, it should be understood that the footprint of the panel 146 may be any shape.

The electrodes 144 may be located in the first lateral widened portion 154, and the impedance sensors 148 may be located in the second lateral widened portion 155. In addition, the conductive leads 150 that are connected to the impedance sensors 148 may extend from the second lateral widened portion 155 through the central narrowed portion 152 to the first lateral widened portion 154. In addition, the central narrowed portion 152 may be wide enough to accommodate the conductive leads 150 extending through the central narrowed portion 152, while the first and second lateral widened portions 154 and 155 may be wide enough to provide enough surface area to stably secure the electrodes 144 and the impedance sensors 148 in place.

The panel or flexible substrate 146 may be positioned so that the first lateral widened portion 154 is positioned on and secured to the anterior scalene muscle 66 so that the phrenic nerve 28 is between the electrodes 144. At the same time, the second lateral widened portion 155 may be positioned at a location that is adjacent to the patient's trachea 84 so that the sensors 148 may be able to receive signals indicative of a condition inside the patient's trachea 84. The second lateral widened portion 155 may be secured to the trachea 84 or any other tissue in the vicinity of the trachea 84.

It is contemplated that the first and second lateral widened portions 154 and 155 may hold the central narrowed portion 152 in place. Thus, the central narrowed portion 152 may not need to be secured to any underlying tissue. Alternatively, it is contemplated that the central narrowed portion 152 may be secured to the underlying tissue in order to provide a more robust securement of the electrode assembly 142.

The electrode assembly 142 may be anchored to the underlying tissue by way of sutures, micro needles, micro-hooks, micro-teeth, a micro-patterned dry adhesive and/or adhesive, or any other biocompatible means of securing the electrode assembly 142 to the underlying tissue. These attachment aids can be biodegradable and eventually bioabsorbed into the body since the assembly in time will be held in place by fibrosis of tissues.

The conductive leads 150 may connect to the wire(s) inside the lead 41 at a perimeter of the first lateral widened portion 154 where the lead 41 terminates. Although FIG. 24 shows the lead 41 attached to the first lateral widened portion 154, the lead 41 may be attached to the panel 146 at a different location. For example, the lead 41 may be attached to the portion of the panel 146 that is closest to the pulse generator 38 so that the path for the lead 41 is minimized. Thus, if the pulse generator 38 is closest to the second lateral widened portion 155, the lead 41 may be attached to the panel 146 at the second lateral widened portion 155 and the conductive leads 150 may be routed toward the second lateral widened portion 155 instead of toward the I first lateral widened portion 154.

In addition, the positioning of the electrodes 144 and the impedance sensors 148 illustrated in FIG. 24 may be used when the right phrenic nerve 28 is being stimulated. If the left phrenic nerve 28 is being stimulated, the positioning of the electrodes 144 and the impedance sensors 148 may be reversed so that the impedance sensors 148 are located at the first lateral widened portion 154 and the electrodes 142 are positioned at the second lateral widened portion 155.

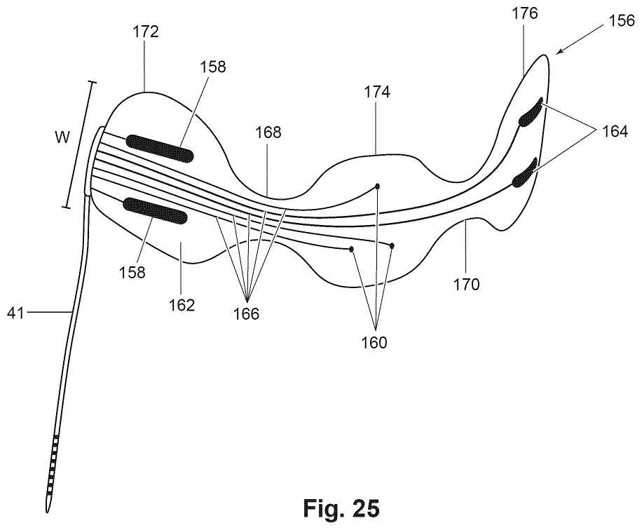

FIG. 25 illustrates another electrode assembly 156 that is configured to stimulate both the left and right phrenic nerves 28 and sense a volume of air inside the patient's airways and lungs. Similar to the electrode assembly 142 illustrated in FIG. 24, the electrode assembly 156 includes a first pair of electrodes 158 and at least one impedance sensor 160 positioned on (or embedded within) a flexible, transparent dielectric panel (substrate) 162. However, the electrode assembly 156 also includes a second pair of electrodes 164 that are positioned on (or embedded within) the flexible panel 162. The second pair of electrodes 164 are located so that the impedance sensors 160 are between the first and second pair of electrodes 158, 164. The impedance sensors 160 may be, for example, tracheal flow impedance sensing leads that are positioned to sense a volume of air inside the patient's airways and lungs. The first and second pairs of electrodes 158, 164 may be, for example, unilateral bipolar electrodes in the form of conductive strips.

Conductive leads 166 may connect the first and second pairs of electrodes 158, 164 and the impedance sensors 160 to the wire(s) inside the lead 41 that extends to the pulse generator 38. It is contemplated that the conductive leads 166 may be positioned on or embedded within the panel 162 and may extend to an edge of the panel 162.

Similar to the panel 146 illustrated in FIG. 24, the panel 162 may include narrowed and widened portions. However, the panel 162 may include additional narrowed and widened portions. In particular, the panel 162 may include a first narrowed portion 168, a second narrowed portion 170, a first widened portion 172, a second widened portion 174, and a third widened portion 176. The first narrowed portion 168 may be positioned between the first and second widened portions 172 and 174. In addition, the second narrowed portion 170 may be between the second and third widened portions 174 and 176. The width W of the panel 162 at the first and second narrowed portions 168, 170 may be less than the width W of the panel 162 at the first, second, and third widened portions 172, 174, and 176. This shape may allow for a more robust securement of the electrode assembly 156, while minimizing the footprint of the electrode assembly 156 to accommodate the small crowded space in the patient's neck 12. However, it should be understood that the footprint of the panel 162 may be any shape.

The first pair of electrodes 158 may be located in the first widened portion 172, and the second pair of electrodes 164 may be located in the third widened portion 176. In addition, the impedance sensors 160 may be located in the second widened portion 174. In addition, the conductive leads 166 that are connected to the impedance sensors 160 and the second pair of electrodes 164 may extend from the third widened portion 176 through the first and second narrowed portions 168, 170 and through the second widened portion 174 to the first widened portion 172. In addition, the first and second narrowed portions 168, 170 may be wide enough to accommodate the conductive leads 166 extending through the first and second narrowed portions 168,170. Because the first narrowed portion 168 includes more conductive leads than the second narrowed portion 170, the width W of the first narrowed portion 168 may be larger than the width W of the second narrowed portion 170. Alternatively, the widths W of the first and second narrowed portions 168, 170 may be the same or the width W of the second narrowed portion 170 may be greater than the width W of the first narrowed portion,

In addition, the first, second, and third widened portions 172, 174, 176 may be wide enough to provide enough surface area to stably secure the first and second pairs of electrodes 158, 164 and the impedance sensors 160 in place. Thus, the widths W of the first, second, and third widened portions 172, 174, 176 may be the same, or different from each other.

The flexible panel 162 may be positioned so that the first widened portion 172 is positioned on and secured to the right anterior scalene muscle 66 so that the right phrenic nerve 28 is between the first pair of electrodes 158. At the same time, the third widened portion 176 may be positioned on and secured to the left anterior scalene muscle 66 so that the left phrenic nerve 28 is between the second pair of electrodes 164. In addition, the second widened portion 174 may be positioned at a location that is adjacent to the patient's trachea 84 so that the sensors 160 may be able to receive signals indicative of a condition inside the patient's trachea 84. The second widened portion 174 may be secured to the trachea 84 or any other tissue in the vicinity of the trachea 84.

It is contemplated that the first, second, and third widened portions 172, 174, 176 may hold the first and second narrowed portions 168, 170 in place. Thus, the first and second narrowed portions 168, 170 may not need to be secured to any underlying tissue. Alternatively, it is contemplated that the first and second narrowed portions 168, 170 may be secured to the underlying tissue in order to provide a more robust securement of the electrode assembly 156.

The electrode assembly 156 may be anchored to the underlying tissue by way of sutures, micro needles, micro-hooks, micro-teeth, a micro-patterned dry adhesive and/or adhesive, or any other biocompatible means of securing the electrode assembly 156 to the underlying tissue. These attachment aids can be biodegradable and eventually bioabsorbed into the body since the assembly in time will be held in place by fibrosis of tissues.

The conductive leads 166 may connect to the wire(s) inside the lead 41 at a perimeter of the first widened portion 172 where the lead 41 terminates. Although FIG. 25 shows the lead 41 attached to the first widened portion 172, the lead 41 may be attached to the panel 162 at a different location. For example, the lead 41 may be attached to the portion of the panel 162 that is closest to the pulse generator 38 so that the path for the lead 41 is minimized. This way, the length of the lead 41 is minimized, does not traverse the patient's midline 93 and remains on the same side of the body as the IPG 38.

Thus, if the pulse generator 38 is closest to the third widened portion 176, the lead 41 may be attached to the panel 162 at the third widened portion 176 and the conductive leads 166 may be routed toward the third widened portion 176 instead of toward the first widened portion 172.

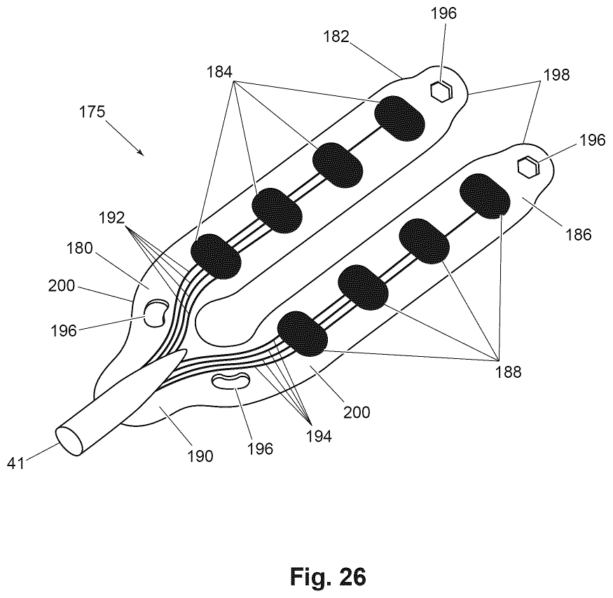

FIG. 26 illustrates another electrode assembly 178. The electrode assembly 178 comprises a Y-shaped, flexible, transparent dielectric panel (substrate) 180. A first branch 182 of the flexible panel 180 may include a first set of electrodes 184, while a second branch 186 of the flexible panel 180 includes a second set of electrodes 188. The electrodes 184 and 188 may be unilateral bipolar electrodes in the form of conductive strips. Both branches 182, 186 may extend from a common base 190. The common base 190 may be connected to the lead 41 that is connected to the IPG 38.

Each electrode 184 on the first branch 182 may be paired with an electrode 188 on the second branch 186 so that one electrode of the pair forms a cathode and the other electrode of the pair forms an anode. When the electrode assembly 178 is positioned on the anterior scalene muscle 66, the phrenic nerve 28 may be located between an electrode 184 and an electrode 188. Each set of electrodes 184, 188 may be arranged in a linear configuration so that if the electrode assembly 178 moves relative to the phrenic nerve 28, the phrenic nerve 28 will still be between two electrodes.

A first set of conductive leads 192 may be positioned on or embedded within the first branch 182 of the panel 180 and may connect the first set of electrodes 184 to the wire(s) inside the lead 41 that extends to the pulse generator 38. In addition, a second set of conductive leads 194 may be positioned on or embedded within the second branch 186 of the panel 180 and may connect the second set of electrodes 188 to the wire(s) inside the lead 41.

Current from the first set of conductive leads 192 may flow from one of the first set of electrodes 184, through the phrenic nerve 28 and adjacent tissue to the electrode 188 from the second set of electrodes that is paired with the first electrode 184. This current flow stimulates the phrenic nerve 28, while the dielectric properties of the flexible panel 180 block current flow away from the phrenic nerve 28. It is contemplated that the configuration may be reversed so that current from the second set of conductive leads 194 flows from one of the second set of electrodes 188, through the phrenic nerve 28 and adjacent tissue and to the electrode 184 from the first set of electrodes that is paired with the second electrode 184.

In addition, each of the first and second branches 182, 186 of the panel 180 may include apertures 196. The apertures 196 may be positioned at strategic locations that may facilitate the anchoring of the electrode assembly 178 to the anterior scalene muscle 66. For example, as illustrated in FIG. 26, the apertures 196 may be located at the distal ends 198 and the proximal ends 200 of the first and second branches 182, 186. The arrangement of apertures 196 is not limited to what is shown in FIG. 26. In particular, the number of apertures 196 may be more or less than what is shown in FIG. 26. For example, the arrangement of apertures 196 may include an aperture 196 at each distal end 198 and an aperture 196 at the base 190. The locations of apertures 196 are only limited to locations that do not interfere with the function of the conductive leads 192, 194 and the function of the electrodes 184, 188.

The apertures 196 may be sized and shaped to receive a fastening device that may be used to secure the electrode assembly 178 to the anterior scalene muscle 66. The fastening device may be, for example, a tack, a staple, or a suture. It should be understood that the other electrode assembly configurations discussed above may also include apertures configured to receive a fastening device. In addition, the apertures 196 may be coplanar with the electrodes 184, 188.

In addition, it is contemplated that the apertures 196 may be replaced with integral (or attached) fasting devices. As discussed above, the fastening devices may be, for example, tacks, staples, or sutures.

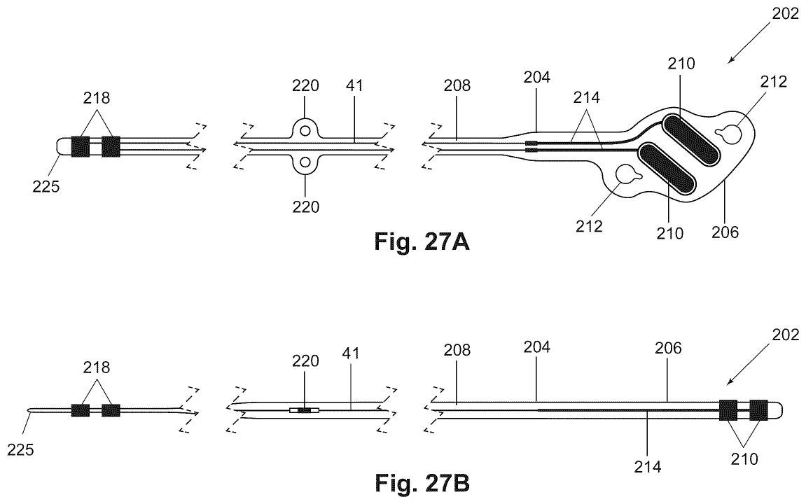

FIGS. 27A and 27B illustrate another electrode assembly 202. FIG. 27A shows a top view of the electrode assembly 202, while FIG. 27B shows a side view of the electrode assembly 202. The electrode assembly 202 may include a flexible, transparent dielectric substrate 204 that includes a paddle-shaped panel portion 206 and a lead portion 208.

The panel portion 206 may be the part of the electrode assembly 202 that is secured to the anterior scalene muscle 66 and may be in the form of a wide and flat membrane. In addition, the panel portion 206 may include a pair of electrodes 210. It is contemplated that the electrodes 210 may be unilateral bipolar electrodes in the form of a conductive strips.

The panel portion 206 may also include apertures 212. The apertures 212 may be positioned at strategic locations that may facilitate the anchoring of the panel portion 206 to the anterior scalene muscle 66. For example, as illustrated in FIG. 26, the apertures 212 may be located at opposite ends of the panel portion 206 so that the electrodes 210 are between the apertures 212. The arrangement of apertures 212 is not limited to what is shown in FIG. 26. In particular, the number of apertures 212 may be more or less than what is shown in FIG. 26. For example, the arrangement of apertures 212 may include only one aperture 212 or more than two apertures 212. The locations of apertures 212 are only limited to locations that do not interfere with the function of the conductive leads 214 and the function of the electrodes 210. In addition, the apertures 212 may be coplanar with the electrodes 210.

The apertures 212 may be sized and shaped to receive a fastening device that may be used to secure the electrode assembly 202 to the anterior scalene muscle 66. The fastening device may be, for example, a tack, a staple, or a suture. In addition, it is contemplated that the apertures 212 may be replaced with integral (or attached) fasting devices. As discussed above, the fastening devices may be, for example, tacks, staples, or sutures.

The panel portion 206 may include conductive leads 214 positioned on or embedded within the panel portion 206. The conductive leads may connect the electrodes 210 to the wire(s) 215 inside the lead portion 208.

The lead portion 208 may be long and thin and may form the lead 41. The lead portion 208 may also include a proximal end 216 with a pair of electrical contacts 218. The electrical contacts 218 may be configured to connect to the IPG 38. The electrical contacts 218 may also be configured to connect to the lead extension 17.

The lead portion 208 may also include strain relief devices (strain relief anchors) 220. As illustrated in FIG. 26, the strain relief device 220 may be in the form of one or more apertures. The lead 41 (or lead portion 208) may receive a suture, tack, or staple to anchor the lead 41 to muscle or connective tissue. Anchoring the lead 41 at the muscle or connective tissue prevents the lead 41 from being pulled out of the IPG 38.

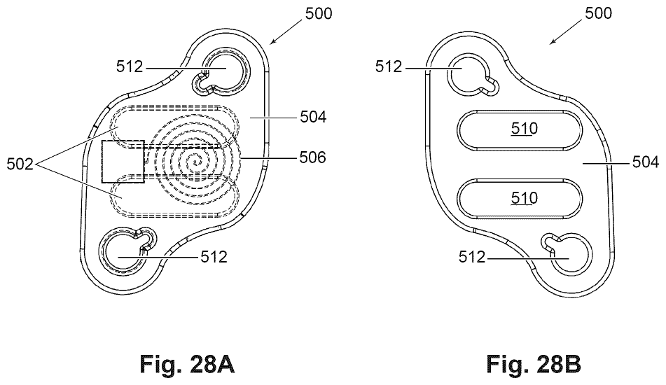

FIGS. 28A and 28B illustrate another embodiment of an electrode assembly 500. FIG. 28A shows the electrode assembly 500 with electrodes 502 embedded in a substrate layer 504 and facing a front face of the substrate layer. FIG. 28B shows just the substrate layer 504, which may be transparent.

The electrodes 502 may be a pair of parallel electrodes arranged to be oriented perpendicularly with respect to a phrenic nerve 28. A back face of the substrate layer 504 supports a spiral antenna 506 configured to receive stimulation signals from a pulse generator external to the patient 10. The pulse generator may be worn, e.g., around the neck, while the patient 10 sleeps and removed while the patient 10 is awake. A controller 508 with an optional rechargeable battery is embedded in the substrate layer 504. The controller 508 may be actuated by a wireless signal received by the antenna to apply electrical energy to the electrodes 502. The battery in the controller 508 may be recharged wirelessly by the pulse generator or other external device.

The substrate layer 504 may be transparent to allow the physician to see the phrenic nerve 28 as the physician uses the delivery tool to position the electrodes 502 over the phrenic nerve 28. The distal end 98, 414 of the delivery tool 16, 400 may also be transparent to allow the physician to see through the distal end 98, 414 and substrate layer 504.

The substrate layer 504 may include openings or recesses 510 to receive the conductive electrodes 502. The substrate layer 504 may also include holes 512 to receive fasteners that secure the electrode assembly 500 to the anterior scalene muscle 66. The holes 512 and fasteners may be configured such that as the fasteners are inserted through the holes 512 and into the muscle so that the fasteners secure the electrode assembly 500 to the muscle.

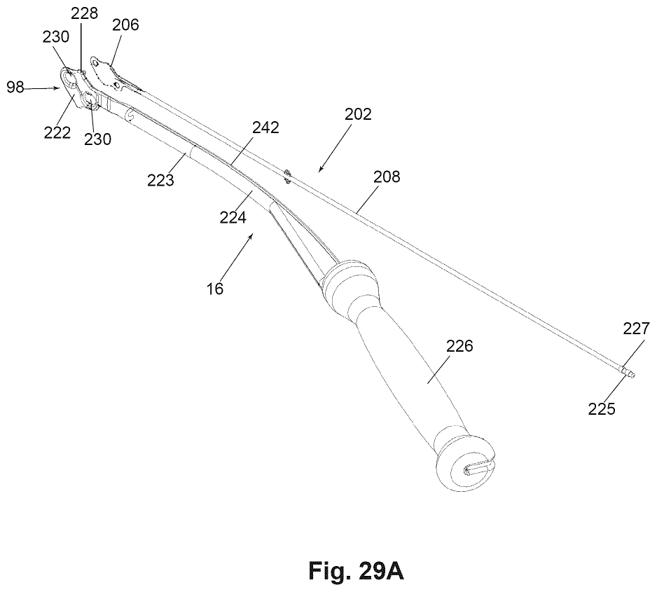

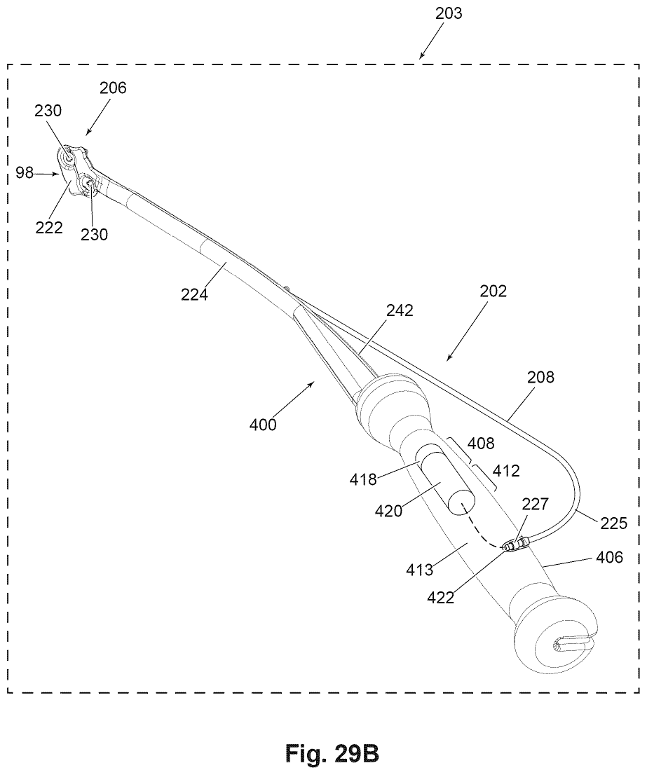

FIG. 29A illustrates an exemplary configuration for the delivery tool 16 for implanting an electrode assembly 202 into the neck 12 of a patient 10.

The delivery tool 16 may include a distal end 98 having an electrode carrier 222, an arm 223 with a channel 242 that receives a lead (or wire), and a handle (or user interface) 226 at a proximal end of the tool 16. The arm 223 may be rigid and may be configured to allow a physician to insert the electrode carrier 222 into a surgical opening 96 in the neck 12 and position the electrode carrier 222 and electrode assembly 202 over the phrenic nerve 28 and against an anterior surface of the anterior scalene muscle 66.

The arm 223 may include a channel 242 extending the length of the arm 223. The channel 242 may be U-shaped in cross section and may be configured to receive the lead (wire) 208. The width of the channel 242 may be sufficient to receive the lead 208. The upper edges of the U-shaped channel 242 may form a gap that is the same as or slightly smaller than the diameter (thickness) of the lead 208. The channel 242 may be configured to releasably hold the lead 208 as the electrode assembly 202 is positioned over the phrenic nerve 28 and secured to the anterior scalene muscle 66. After the electrode assembly 202 is fastened to the anterior scalene muscle 66, the lead 208 may be pulled by the physician out of the channel 242 to remove the electrode assembly 202 from the electrode carrier 222 without dislodging the electrode assembly 202 from the anterior scalene muscle 66.

FIG. 29A shows the delivery tool 16 separate from the electrode assembly 202. The delivery tool 16 and electrode assembly 202 may be assembled and packaged in a sterile package 203 (see FIG. 29B) to be opened by a physician or other member of the surgical team. The assembly may include the electrode panel 206 seated in a recessed receiving space 228 of the electrode carrier 222 of the delivery tool 16, and the lead 208 seated in the channel 224 of the delivery tool. As shown in FIG. 29A, a proximal end 225 of the electrode assembly 202 may include a connector 227 configured to connect to a source of electrical current and receive signals to be delivered to the electrode panel 206 and stimulate the phrenic nerve 28. The source of electrical current may be the external pulse generator 18 shown in FIG. 1.

FIG. 29B illustrates an exemplary configuration for a delivery tool 400 for implanting the electrode assembly 202 into the neck 12 of a patient 10. The delivery tool 400 may be similar to the delivery tool 16. In addition, the delivery tool 400 may include a handle 410 that includes an internal battery 418 and an electronic circuit 420. The internal battery 418 and electronic circuit 420 may function as a source of electrical current pulses to be delivered to the electrode panel 206.

An electrical connector 422 may provide a temporary electrical connection between the battery 418 and electronic circuit 420 and the connector 227 at the proximal end 225 of the lead 208. The proximal end 225 of the lead 208 may be connected to the electrical connector 422 and the lead 208 and electrode panel 206 may be seated in the delivery tool 400 before being packaged into the sterile package 203.

The assembly of the delivery tool 400 and electrode assembly 202 may be removed from the sterile package 203 in a sterile environment that includes the patient's neck 12 during the surgery. The physician may hold the handle 413 to maneuver the electrodes in the panel portion 206 over the phrenic nerve 28 and the anterior surface of the anterior scalene muscle 66. To confirm that the electrodes are positioned over the phrenic nerve 28, the physician may actuate a controller by way of, for example, a button 408 on the handle 413 of the delivery tool 400 to activate the electronic circuit 420 and apply electrical power pulses to the electrodes. A light, audible speaker, vibrator, or other indicator 412 may indicate to the physician that electrical power is being applied to the electrode. The physician may monitor the patient to determine if the electrical current delivered by the electrodes is stimulating the phrenic nerve 28. Once there is confirmation that the electrical current is stimulating the phrenic nerve 28, the panel 206 may be secured to the anterior scalene muscle 66 using the fixation tool 20 (see FIG. 17). Then, the panel 206 may be detached from the carrier portion 222 of the delivery tool 400 by pulling the carrier portion 222 away from the anterior scalene muscle 66. Also, the lead portion 208 of the electrode assembly 202 may be pulled from the channel 242 of the arm 223 of delivery tool 400 as the delivery tool 400 is pulled away from the anterior scalene muscle 66 and out of the surgical opening 96 in the neck 12. Further, the connector 227 at the proximal end 225 of the lead portion 208 may be disconnected from the connector 422 on the handle 413 or from the external pulse generator 18 (See FIG. 1).

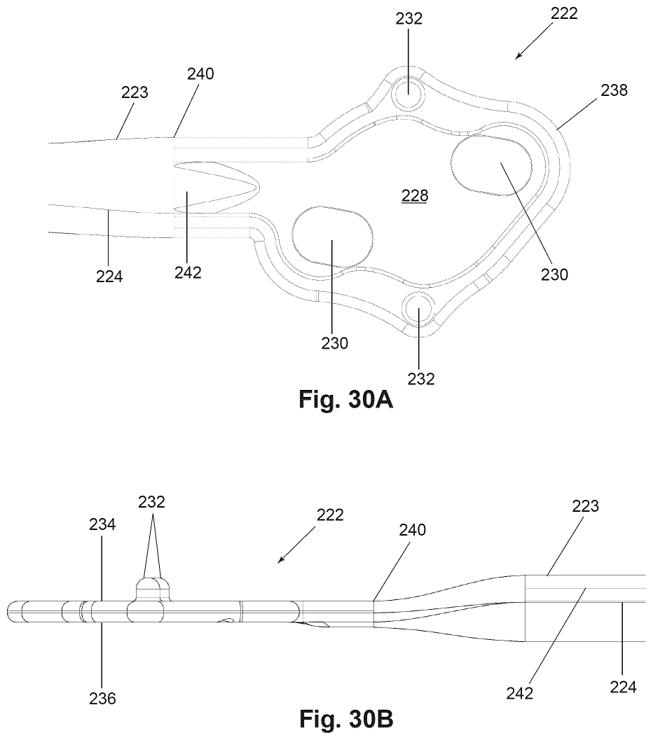

FIGS. 30A and 30B illustrate the electrode carrier 222 on a distal end of the arm 223 of the delivery tool 16, 400. FIG. 31 illustrates an electrode panel 206 seated in the electrode carrier 222.

The electrode carrier 222 may include a recessed receiving space 228, apertures 230 within the space 228, and a detents 232 on opposite sides of the receiving space 228. In addition, the electrode carrier 222 may have a front side 234 that faces the anterior scalene muscle 66 and the phrenic nerve 28 when implanting the electrode assembly 202. A rear side 236 of the electrode carrier 222 may face away from the anterior scalene muscle 66 and the phrenic nerve 28 when implanting the electrode assembly 202.

The receiving space 228 may be shaped to receive the panel portion 206 of the electrode assembly 202 and is bound by a rim 238. The perimeter of the receiving space 228 may be shaped complimentarily to the perimeter of the panel portion 206 (i.e., the perimeter of the receiving space 228 may have the same shape as the perimeter of the panel portion 206). In addition, the size of the receiving space 228 may be the same as or slightly larger than the size of the panel portion 206 so that the panel portion 206 fits snuggly in the receiving space 228. This way, the panel portion 206 may be held in place in the receiving space 228 by friction. In addition, the shape of the perimeter of the receiving space 228 may prevent the panel portion 206 from shifting while in the receiving space 228.

The panel portion 206 may be held within the receiving space 228 by other means. For example, the receiving space 228 and/or the panel portion 206 may be coated with a weak adhesive. Alternatively, the panel portion 206 may be held within the receiving space 228 by way of a hook and loop connection, a clip, or any other means. The force holding the panel portion 206 to the receiving space 228 may be weaker than the force that the fasteners apply to hold the panel portion 206 in place on the anterior scalene muscle 66.

The shape of the perimeter of the receiving space 228 may be the same or different from the perimeter of the panel portion 206 and/or may be large enough so that there is a gap between the panel portion 206 and the rim 238 when the panel portion 206 is positioned in the receiving space 228.

The apertures 230 may be positioned within a floor of the receiving space 228. In addition, the apertures 230 may be positioned to align with the apertures 212 in the panel portion 206 of the electrode assembly 202 when the panel portion 206 is positioned within the receiving space 228. The alignment between the apertures 230 and the apertures 212 allows for the electrode assembly 202 to be permanently secured to the anterior scalene muscle 66 while the electrode assembly 202 is being held in place by the delivery tool 16, 400.

In addition, the apertures 230 in the receiving space 228 may be larger than the apertures 212 in the panel portion 206 of the electrode assembly 202. In particular, the apertures 230 may be large enough so that the fastening device that permanently secures the electrode assembly 202 to the anterior scalene muscle 66 can be inserted through the aperture 230 without engaging the electrode carrier 222. This way, the fastening device can be inserted through both the apertures 212 and 230 but only fasten the electrode assembly 202 to the anterior scalene muscle 66, which allows the delivery tool 16 to hold the electrode assembly 202 in place while the electrode assembly 202 is being permanently secured and then be removed from the electrode assembly 202 once the electrode assembly 202 is permanently secured in place.

The detents 232 may be positioned on the rim 238 on opposite sides of the electrode carrier 222. The detents 232 may project from the rim 238 to temporarily hold the electrode carrier 222 and the panel portion 206 of the electrode assembly 202 in place while a test stimulation current is being discharged to the phrenic nerve 28. The detents 232 may press into the anterior scalene muscle 66 but do not puncture the muscle. By pressing into the muscle, the detents 232 aid in holding the position of the electrode carrier 222 and panel portion 206 of the electrode assembly 202 over the phrenic nerve 28 as the fixation tool 20 (or the surgeon) inserts the fixation devices, e.g., screws, through the apertures 212 in the panel portion and the apertures 230 in the receiving space 228, and into the anterior scalene muscle. The detents 232 may have a rounded engagement surface so that the detents 232 do not puncture or damage the anterior scalene muscle 66 while securing the electrode assembly 202 in place during testing. Also, the rounded engagement surface allows the delivery tool 16, 400 to be easily removed from the anterior scalene muscle 66 to reposition the electrode assembly 202 at another location on the anterior scalene muscle 66.

The electrode carrier 222 may be arranged so that the front side 234 of the receiving space 228 is recessed and the detents 232 project from the front side 234 of the electrode carrier 222. This way, the electrode assembly 202 is between the floor of the receiving space 228 and the anterior scalene muscle 66 and the phrenic nerve 28 and will not interfere with the removal of the delivery tool 16 from the surgical opening 96.

The electrode carrier 222 may be integrally formed with the lead carrier 224. At the very least, the electrode carrier 222 and the lead carrier 224 may both be formed from transparent material so that the delivery tool 16, 400 does not interfere or minimally interferes with the surgeon's view of the phrenic nerve 28 and the anterior scalene muscle 66. It is also contemplated that the electrode carrier 222 and/or the lead carrier 224 may be made from flexible material and may be flexible.

The electrode carrier 222 may be pivotable relative to the lead carrier 224. For example, the electrode carrier 222 may be allowed to flex (due to the flexible material) relative to the lead carrier 224. Alternatively, the electrode carrier 222 may include a hinge structure (e.g., a living hinge) at a transition point 240 between the electrode carrier 222 and the lead carrier 224. Allowing the electrode carrier 222 to pivot relative to the lead carrier 224 makes the delivery tool 16, 400 more versatile. Each patient has a unique anatomy, and the anatomical arrangement in different patients' necks may vary. A pivotable delivery tool 16, 400 can accommodate the different anatomical arrangements that would be expected in different patients 10.

The receiving space 228 may transition into a channel 242 that extends the length of the lead carrier 224. The channel 242 provides at least part of a path for the lead 41 and may extend parallel to the central longitudinal axis of the delivery tool 16, 400. It is contemplated that the width and depth of the channel 242 may be marginally larger than the diameter of the lead 41 so that the channel 242 can accommodate the lead 41. It is further contemplated that the channel 242 may be sized to allow the lead 41 to slide along the length of the channel 242, especially when the delivery tool 16, 400 is being separated from the electrode assembly 202 so that the delivery tool 16, 400 does not pull on the lead 41 and does not pull the electrode assembly 202 away from the phrenic nerve 28.

The lead carrier 224 may be bent along an axis that is parallel to the central longitudinal axis. In addition, the lead 41 may be allowed to separate from the channel 242 at the bend so that the lead 41 can be connected to the lead extension 17 and/or the external current generator 18. In addition, the height H of the delivery tool 16, 400 may be greater at the lead carrier 224 than at the electrode carrier 222.

It is contemplated that the channel 242 may extend at least part way into the handle 226, 406. The handle 226, 406 may be the portion of the delivery tool 16, 400 that is held by the user. The handle 226, 406 may include an ergonomic grip (not shown) that may make it easier to hold. In addition, the handle 226, 406 may be more rigid than the other portions of the delivery tool. For example, the handle may be formed from a rigid material. It is also contemplated that the handle 226, 406 may be formed from a flexible or compressible material for comfort. Alternatively, the handle 226, 406 may have a rigid core surrounded by a more flexible or compressible material for comfort.

The delivery tool 16, 400 may also include a visual nerve alignment feature 244 at the transition point 240. The nerve alignment feature 244 may allow the surgeon to visually align the electrode assembly 202 with the phrenic nerve 28 during installation. The visual nerve alignment feature 244 may be raised features (e.g., bumps), notches (or indentations), printed marking, or any other means for providing a visual aid for aligning the electrode assembly 202.

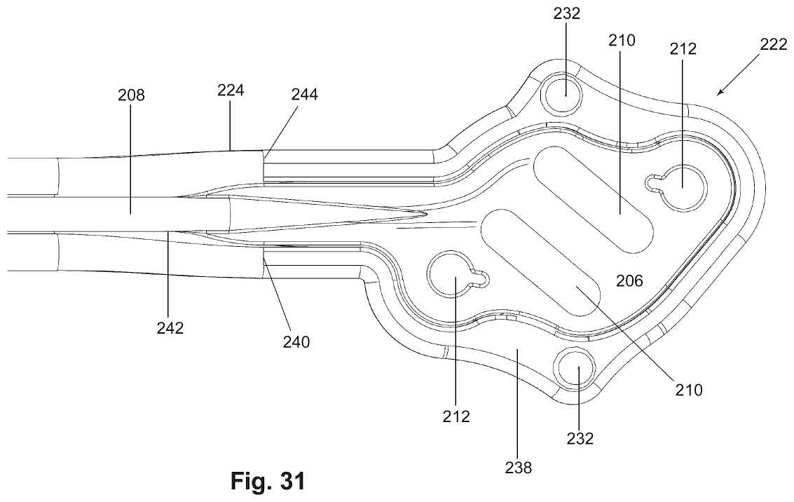

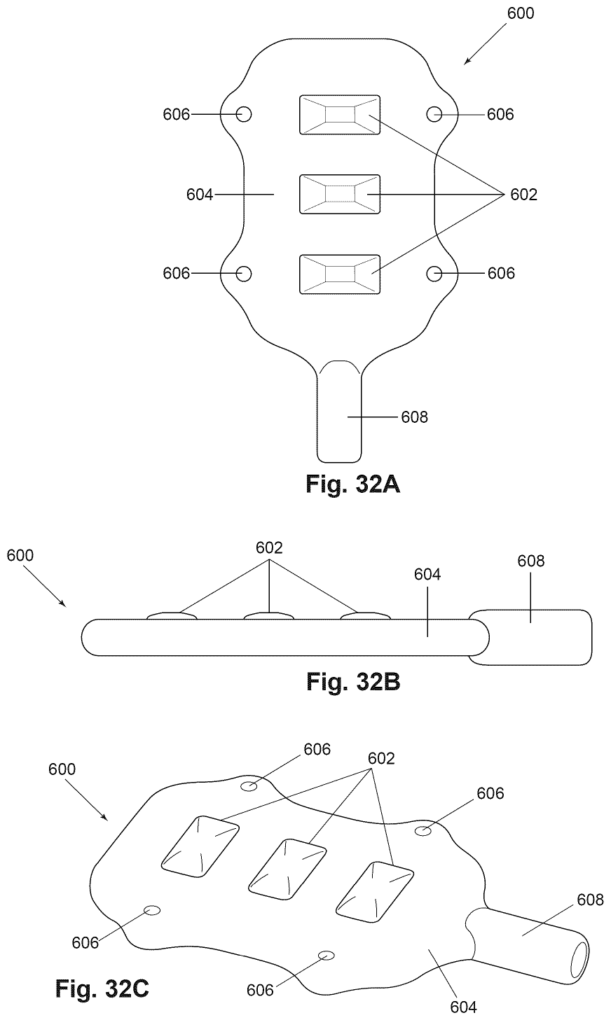

FIGS. 32A-C illustrate another embodiment of an electrode assembly 600. The electrode assembly 600 includes electrodes 602 that are at least partially embedded within a substrate layer 604 and facing a front face of the substrate layer 604. The electrodes 602 are shown with a portion that stands proud of the substrate layer 604. This may facilitate better connection between the phrenic nerve 28 and the electrodes 602. It is contemplated that the entirety of the electrodes 602 may be positioned on top of the substrate layer 604 and that no part of the electrodes 602 are embedded within the substrate layer 604. It is further contemplated that some of the electrodes 602 may be embedded within the substrate layer 604 and that some may be positioned on top of the substrate layer 604. Also, the distance by which the electrodes 602 extend from the surface of the substrate layer 604 may be different for each electrode 602. In addition, although this configuration includes three electrodes 602, the electrode assembly 600 may include more or less electrodes 602.

The electrodes 602 may be aligned in a row on the substrate layer as illustrated or may form a different pattern. In addition, the substrate layer 604 may be transparent to allow the physician to see the phrenic nerve 28 as the physician uses the delivery tool to position the electrodes 602 over the phrenic nerve 28.

The substrate layer 604 may include holes 606 to receive fasteners that secure the electrode assembly 600 to the anterior scalene muscle 66. The holes 606 and fasteners may be configured such that as the fasteners are inserted through the holes 606 and into the muscle so that the fasteners secure the electrode assembly 600 to the muscle.

In addition, the electrodes 602 may be electrically connected to a lead 608 located at one end of the substrate layer 604. The lead 608 may be connected to a pulse generator.

While at least one exemplary embodiment of the present invention(s) is disclosed herein, it should be understood that modifications, substitutions, and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1.-17. (canceled)

18. The delivery tool of claim 30, further comprising a visual nerve alignment feature including a marking or a raised features or a notch or an indentation providing a visual aid for aligning the electrode assembly.

19. The delivery tool of claim 30, wherein the electrode carrying portion is transparent or wherein the lead carrying portion is transparent or wherein both the electrode carrying portion and the lead carrying portion are transparent.

20.-29. (canceled)

30. A delivery tool configured to position an electrode assembly in a patient's neck, the delivery tool comprising:
an electrode carrying portion including a receiving space with at least one aperture in a floor of the receiving space, the receiving space is configured to hold the electrode assembly;
a lead carrying portion extending from the electrode carrying portion, the lead carrying portion configured to hold a lead connected or connectable to the electrode assembly, and
a cable segment integrated in the delivery tool and comprising a first connector for connection with the electrode assembly and a second connector for direct connection with an electric current generator connection or with a further cable segment connectable to the electric current generator,
wherein the current generator is either integrated in the delivery tool.

31. The delivery tool of claim 30, wherein the cable segment is integrated in the lead carrying portion and defines the lead connected or connectable to the electrode assembly.

32. The delivery tool of claim 30, wherein the electric current generator is integrated into the delivery tool, optionally housed in the/a handle of the delivery tool.

33. The delivery tool of claim 30, wherein the electric current generator comprises a battery, and electronic circuitry configured to deliver electrical current through said lead to the electrode assembly mounted on a distal end of the delivery tool.

34. The delivery tool of claim 33, further comprising a user interface including a touch sensitive item on the handle, communicatively connected to the electronic circuitry and configured to be operated by a user to issue a command for the electronic circuitry,
wherein the electronic circuit is configured to receive said command and upon receipt of said command control the electric current generator to deliver electrical current to the electrode assembly to stimulate the phrenic nerve during the placement and/or securement of the electrode assembly.

35. The delivery tool of claim 30, wherein the lead is configured to be detached from the delivery tool and electrically coupled to a pulse generator that may be implanted in the patient or positioned next to the patient.

36. The delivery tool of claim 30, wherein the delivery tool is disposable.

37. The delivery tool of claim 30, wherein the delivery tool is packaged in a sterile kit ready to be used by a physician.

38.-139. (canceled)

140. A delivery tool configured to position an electrode assembly in a patient's neck, the delivery tool comprising:
an electrode carrying portion including a receiving space with at least one aperture in a floor of the receiving space, the receiving space is configured to hold an electrode assembly;
a lead carrying portion extending from the electrode carrying portion, the lead carrying portion configured to hold a lead connected or connectable to the electrode assembly;
an electric current generator with an electrical connector configured to be connected to a lead with electrodes in the electrode assembly;
a grip portion configured to be gripped by a user, wherein the delivery tool is configured to be held by the user when the user at least at the grip portion, and,
an indicator configured to provide a signal to the user representing a condition and/or position of an electrode positioned on the electrode carrying portion.

141. The delivery tool of claim 140, wherein the indicator includes at least one of a light, an audible speaker, and a vibrator.

142. The delivery tool of claim 140, wherein the signal is representative of an amount of power applied to the electrode. indicate to the physician that electrical power is being applied to the electrode and/or whether the electrical current delivered by the electrodes is stimulating the phrenic nerve.

143. The delivery tool of claim 140, wherein the indicator is located on or in the grip portion.

144. The delivery tool of claim 140, wherein the electric current generator is positioned within the grip portion.

145-152. (canceled)

153. The delivery tool of claim 140, further comprising a visual alignment feature configured to aid in the alignment of the electrode assembly with a stimulation target.

154. (canceled)

155. The delivery tool of claim 140, wherein the electrode carrying portion is transparent or wherein the lead carrying portion is transparent or wherein both the electrode carrying portion and the lead carrying portion are transparent.

155. (canceled)

156. (canceled)

157. (canceled)

158. The delivery tool of claim 140, wherein a distal end of the delivery tool includes an integrated neuromonitoring nerve detecting tip configured for emitting a signal indicating close electric contact between the detecting tip and a patient's neuronal structure.

159. A method to implant an electrode assembly in a patient's neck, the method comprising:
surgically creating an opening in the neck to expose the phrenic nerve extending over an anterior scalene muscle in the neck;
releasably mounting an electrode assembly to an electrode carrying portion at a distal portion of a delivery tool, wherein the delivery tool includes a handle and a lead carrying portion extending from the electrode carrying portion to the handle;
manipulating the delivery tool to position the electrode assembly mounted to the electrode carrying portion at the phrenic nerve proximate to the anterior scalene muscle, while a conductive lead extends from the electrode assembly, along the lead carrying portion and to an electrical current generator within the handle;
an electric current generator with an electrical connector configured to be connected to a lead with electrodes in the electrode assembly;
actuating the electric current generator to apply current through the lead to the electrode assembly while the electrode assembly is at the phrenic nerve;
while the electric current generator is applying the current to the electrode assembly, adjusting the position of the electrode assembly relative to the phrenic nerve, and
based on a determination that the phrenic nerve is stimulated by the current, fixing the position of the electrode assembly relative to the phrenic nerve and releasing the electrode assembly from the electrode carrying portion and removing the delivery tool from the opening in the neck.

160. The delivery tool of claim 140, further comprising a securing device configured to hold the electrode assembly in place relative to a stimulation target when the delivery tool is inserted into a patient's body, the securing device being further configured to allow the electrode to be repositioned after the electrode assembly has been held in place by the securing device.
